SEQUENCE LISTING (77 sequences)

SEQ ID NO: 1 - DNA, 52 bp, Homo sapiens
aagggaggggagggggccccgacggaggccacagtaaaggagtggaggggcc

SEQ ID NO: 2 - DNA, 52 bp, Homo sapiens
tgcaattcaaaatcaagggctgcttctgaggaggcctctccaccgggctgct

SEQ ID NO: 3 - DNA, 52 bp, Homo sapiens
tgccggaggccccgcctctgctgttgtgcagctgtgttgctgggcctggtgc

SEQ ID NO: 4 - DNA, 52 bp, Homo sapiens
tccgtgtctgcttctgtattttgtgctggttttagaggatccctgggctgcc

SEQ ID NO: 5 - DNA, 52 bp, Homo sapiens
cccggcccccctctcgggctctctcatcccagcctggtactgaaggtgccag

SEQ ID NO: 6 - DNA, 52 bp, Homo sapiens
tggccccaggggctaggcacaggcgctggtgccgtggcctagtgaggagcac

SEQ ID NO: 7 - DNA, 52 bp, Homo sapiens
cgagcagaaggagtgggccatgaacagtgggcgacaagaaaggtggggtccg

SEQ ID NO: 8 - DNA, 52 bp, Homo sapiens
gagacaaattagaaatgtcagtctgagagagagtggtaggtagccagatact

SEQ ID NO: 9 - DNA, 52 bp, Homo sapiens
tgttagccccatggggacgactgccaggcctgggaaacgaagaggagtcagc

SEQ ID NO: 10 - DNA, 52 bp, Homo sapiens
accggtacctgccggacacgcttctctttggaggtgagccccaaccaggatg

SEQ ID NO: 11 - DNA, 52 bp, Homo sapiens
cttccctgttctcttctgctctgtctctctggtgccctgaggctggcctcca

SEQ ID NO: 12 - DNA, 52 bp, Homo sapiens
agtgaggatgcagtgctggtttctgtcccacctacacctagagctgtcccca

SEQ ID NO: 13 - DNA, 52 bp, Homo sapiens
ttgggacacccaccctcacggcctcctccacctggtgctcgctcacctgcag

SEQ ID NO: 14 - DNA, 52 bp, Homo sapiens
aaactggacactgctgttagcagccaggactaggagcacgaggggcacagcc

SEQ ID NO: 15 - protein, 52 aa, Homo sapiens
GAAATGCTGTCAGAAGCCTACCCCACTGGTACCTGTCATGGGCCTTTTCATT

SEQ ID NO: 16 - DNA, 52 bp, Homo sapiens
gggacaggcgggcactgggtgcctcctttgcaccagccaggcccagcctgca

SEQ ID NO: 17 - DNA, 52 bp, Homo sapiens
ctgcgtccggccgtattccagctttctaaaacaacaaaaaacaacaaaaact

SEQ ID NO: 18 - DNA, 52 bp, Homo sapiens
gggttctgtgagcatcggaggcacgagggggtgaggggctcaggagcaggtt

SEQ ID NO: 19 - DNA, 52 bp, Homo sapiens
ctcctctgacactgtcgcttctccatcggcattagattttcagtcctgctca

SEQ ID NO: 20 - DNA, 52 bp, Homo sapiens
ttacaggagaagctgttatcaccccagtttccagggggctgggaaccctggg

SEQ ID NO: 21 - DNA, 52 bp, Homo sapiens
gaggtttctcccgatagtggaccccaccgggccatgccctatggctgcgtgt

SEQ ID NO: 22 - DNA, 52 bp, Homo sapiens
tggggagccagctgcctgtgcaggtagcaaaatgggtggcagaagtggggtg

SEQ ID NO: 23 - DNA, 52 bp, Homo sapiens
ccatgcaggtctggcccatggaaggcgaggggagggggccccgacggggcca

SEQ ID NO: 24 - DNA, 52 bp, Homo sapiens
cggactaggagcacgaggggcacagcgccccatgcctggctaggtagggccg

SEQ ID NO: 25 - DNA, 52 bp, Homo sapiens
cgtgggcgacaagaaaggtggggtcctgggccagcaggtgctcagctctggg

SEQ ID NO: 26 - DNA, 52 bp, Homo sapiens
agccactgctcgtgcaaagaaagcactgtgtctcctgcagatcttcctcctg

SEQ ID NO: 27 - DNA, 52 bp, Homo sapiens
tcctctggagtcccagggtgccagggttccctgatgacccctgcaggccctg

SEQ ID NO: 28 - DNA, 52 bp, Homo sapiens
ctgggcccaagcaatcctcccacttctagcctcctgagtagctgcagctata

SEQ ID NO: 29 - DNA, 52 bp, Homo sapiens
gctctgggcccagcctgctctcccccgaagcaagccactgctcgtgcaaaga

SEQ ID NO: 30 - DNA, 52 bp, Homo sapiens
gccttctgctgtctcttctgaggccaggaaaggcgaagcactgccctcgccc

SEQ ID NO: 31 - DNA, 52 bp, Homo sapiens
gagctggagggggggtcttcctgggctggcctgtgtagcagcagctccaggc

SEQ ID NO: 32 - DNA, 52 bp, Homo sapiens
ctctgggacagggacccaggaccaggtcatcaaagcccttacaggagaagct

SEQ ID NO: 33 - DNA, 52 bp, Homo sapiens
atctggatttgatggcatttttattagtggtgttgttttagagacagtgtct

SEQ ID NO: 34 - DNA, 52 bp, Homo sapiens
gcagcaccagcccctctgggccccactgtggcacagagtggaagcatctcct

SEQ ID NO: 35 - DNA, 52 bp, Homo sapiens
agaggcaatggctcagaccagctccctgcatccctgtagttgcctccctggc

SEQ ID NO: 36 - DNA, 52 bp, Homo sapiens
ttgcttacagaagaggcaatggctcacgaccagctcccgcatccctgtagtt

SEQ ID NO: 37 - DNA, 52 bp, Homo sapiens
gggctctggcctttggccttttcctcgtgagctgcatgttggccactgtgac

SEQ ID NO: 38 - DNA, 52 bp, Homo sapiens
ctgaaaacatcttgtaagcttttttatggccaatatattatcaaacgcaatt

SEQ ID NO: 39 - DNA, 52 bp, Homo sapiens
ggaccttggtcatcgccaggttaggagtttatgttgatgattatccaaaggc

SEQ ID NO: 40 - DNA, 52 bp, Homo sapiens
ctggggacaggtggaccccgcagcaagcaccagcccctctgggccccatgtg

SEQ ID NO: 41 - DNA, 52 bp, Homo sapiens
gtgaagtgatctgacgttgggtgggaggtctccggacttggggtggggaatc

SEQ ID NO: 42 - DNA, 52 bp, Homo sapiens
gagggccttggtgacttctccaacaaggctccctgttggaggccttggggta

SEQ ID NO: 43 - DNA, 52 bp, Homo sapiens
tacagcctctcctttggccacccgtcgactacccccaactccgggccatggg

SEQ ID NO: 44 - DNA, 52 bp, Homo sapiens
actgggatagggtgtggggaattcgagaccgctgtgaagtgatctgacgttg

SEQ ID NO: 45 - DNA, 52 bp, Homo sapiens
ccaaggagcagcgcatcctgaaccactgtgctgcagcatgcggagcccggga

SEQ ID NO: 46 - DNA, 52 bp, Homo sapiens
tggtttgtgtatgttcttggtaaactcagcccttggtcttacacatcatttc

SEQ ID NO: 47 - DNA, 52 bp, Homo sapiens
ttcagtcagcctcagcctctccaaacgagccaggcattccagtagagccctg

SEQ ID NO: 48 - DNA, 52 bp, Homo sapiens
cccagaccagacaccagggcagaaactggcacaggaccaaggagatggggtg

SEQ ID NO: 49 - DNA, 52 bp, Homo sapiens
ccagcggatggtggatttcgctggcagtgaaggacaaggtgtgcatgcctga

SEQ ID NO: 50 - DNA, 52 bp, Homo sapiens
gcctgctgtcaccaggggcgaggctcgatcaccatcgagatcaaccccgact

SEQ ID NO: 51 - DNA, 52 bp, Homo sapiens
accatcgagatcaaccccgactgtgctcgccatcacccagcggatggtggat

SEQ ID NO: 52 - DNA, 52 bp, Homo sapiens
tgccatggtcggatgagagcagccactggacaatgtctgtgcaattgtgcgt

SEQ ID NO: 53 - DNA, 52 bp, Homo sapiens
aaagttacgcttaataatgaatgtttgcagcactttcttctcttcaggtatt

SEQ ID NO: 54 - DNA, 52 bp, Homo sapiens
gttgaaagttactgaaaacatcttggtaagcttttttaggccaatatattat

SEQ ID NO: 55 - DNA, 52 bp, Homo sapiens
gtaagcaaacccttttctgatagcagtagataggcaagcatcctatgaggtt

SEQ ID NO: 56 - DNA, 52 bp, Homo sapiens
tccccaaccctgcacaggcaagatcgttggacgccgtgattcaggagcacca

SEQ ID NO: 57 - DNA, 52 bp, Homo sapiens
gctgcagcatgcggagcccgggaacgtcacagagcgtgctggaggccattga

SEQ ID NO: 58 - DNA, 52 bp, Homo sapiens
gcaccagagggcacgagaaggctggcttccctggcgctgacacgtcaggcaa

SEQ ID NO: 59 - DNA, 52 bp, Homo sapiens
gcatttctgaaccttgcccctctgcgaaacacaagggggcgatggtggcact

SEQ ID NO: 60 - DNA, 52 bp, Homo sapiens
ctgaggcactggggctggggcctgtgcccttatcggctggaacgagttcatc

SEQ ID NO: 61 - DNA, 51 bp, Homo sapiens
tcctgcagcccatccacaacctgctacatgggtgacaccaaggagcagcgc

SEQ ID NO: 62 - DNA, 52 bp, Homo sapiens
gttgctttttattgtttaaagtgacctctccaagcctgagggaccgtaggag

SEQ ID NO: 63 - DNA, 52 bp, Homo sapiens
gctttttggatttttccagccagggagtttttgtgtcctgttgctttttatt

SEQ ID NO: 64 - DNA, 52 bp, Homo sapiens
gctaacagacctgctttttggatttcttccagccagggatttttgtgtcctg

SEQ ID NO: 65 - DNA, 52 bp, Homo sapiens
actgtgtgctccggttgttgcatccagagggttgatcggatggtggttccca

SEQ ID NO: 66 - DNA, 52 bp, Homo sapiens
cagggccccaaggtgggcggttcgggtgattcagagagggcagctctgtgtt

SEQ ID NO: 67 - DNA, 52 bp, Homo sapiens
agcatgcggagcccgggaacgcacaagagcgtgctggaggccattgacacct

SEQ ID NO: 68 - DNA, 52 bp, Homo sapiens
tgtgaggcactgaggatgccctcacgacgtgcatctgcatgtggcgtgcatg

SEQ ID NO: 69 - DNA, 52 bp, Homo sapiens
accactggaaggaccggtacctgccaggacacgcttctcttggaggtgagcc

SEQ ID NO: 70 - DNA, 52 bp, Homo sapiens
gcccatcccagaggcatgtggggtcaggataccagtgtttcaaggcacctgc

SEQ ID NO: 71 - DNA, 52 bp, Homo sapiens
aaatcccctagaagcctggtgtccgctatgacctccccctagggcggagcct

SEQ ID NO: 72 - DNA, 52 bp, Homo sapiens
ggtagggccgctggaccctggacacaggattggaaggaaccagcactagcag

SEQ ID NO: 73 - DNA, 52 bp, Homo sapiens
aacccctctccttgggtgcctctcccttcataggcctgagttcctggcactg

SEQ ID NO: 74 - DNA, 52 bp, Homo sapiens
cctgacatgctaacctctctgaactagcaacactggattgttcttttttaag

SEQ ID NO: 75 - DNA, 52 bp, Homo sapiens
gtggttactttctggagagagcatgtcggcatgcaggagctggagggggggt

SEQ ID NO: 76 - DNA, 1610 bp, Homo sapiens
gcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagaggatccctgggctgcctggggaagcacccagggccagggagtgtgaccctgcaggctccacacaggactgccagaggcacacacctgctctgtctacccgagggcaccagagggcacgagaaggctggctccctggcgctgacacgtcaggcaactgaggcacaaggctggcatttctgaaccttgcccctctgcaaacacaagggggcgatggtggcactccaagcaaaggggcgtgtgggtgctgcaggaggagcacagagcactggcgcccctcccctcccgccctgcagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataa

SEQ ID NO: 77 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

(19) United States
(12) Patent Application Publication
(10) Pub. No.: US 2015/0110718 A1
(43) Pub. Date: Apr. 23, 2015

(54) METHODS AND KITS FOR DETERMINING A PLACEBO PROFILE IN SUBJECTS FOR CLINICAL TRIALS AND FOR TREATMENT OF PATIENTS

(71) Applicant: Biometheus LLC, Naples, FL (US)

(72) Inventors: Gunther Winkler, Naples, FL (US); Kathryn T. Hall, Jamaica Plains, MA (US); Ted J. Kaptchuk, Cambridge, MA (US)

(21) Appl. No.: 14/516,523

(22) Filed: Oct. 16, 2014

Related U.S. Application Data
(60) Provisional application No. 61/891,975, filed on Oct. 17, 2013.
(60) Provisional application No. 61/891,973, filed on Oct. 17, 2013.

(51) Int. Cl. C12Q 1/68; A61K 31/135; A61K 31/70; A61K 49/00

(52) U.S. Cl. CPC C12Q 1/6876; A61K 49/0004; A61K 31/135; A61K 31/70; C12Q 2600/156
USPC 424/9.2; 506/9; 506/2; 435/7.4; 435/15; 435/6.11; 514/657; 514/23

(57) ABSTRACT

The present invention is directed to methods and assays for identifying subjects participating in clinical trials that may exhibit a placebo response and identifying treatments for subjects with varying degrees of placebo responses. In one aspect, a method of selecting subjects to participate in a clinical trial is disclosed. In another aspect, methods for treating a subject and determining a treatment dosage are disclosed. In an exemplary embodiment, a method for determining a response to a treatment of a subject having, suspected of having, or at risk for developing a disorder, such as cardiovascular disorder, irritable bowel syndrome, diabetes, autoimmune disorders, inflammation, neurological disorders, chronic pain, cancer, cancer treatments, allergies, depression, migraines, addiction, obesity, and other disorders, syndromes, or diseases, is disclosed.

### RELATED APPLICATIONS

This patent application claims the benefit of U.S. Provisional Patent Application No. 61/891,973 filed on Oct. 17, 2013, entitled METHODS AND KITS FOR DETERMING A PLACEBO PROFILE IN SUBJECTS FOR CLINICAL TRIALS, naming Gunther Winkler, Kathryn T. Hall, and Ted J. Kaptchuk as inventors and claims the benefit of Provisional Patent Application No. 61/891,975 filed on Oct. 17, 2013, entitled METHODS AND KITS FOR DETERMING A PLACEBO PROFILE IN SUBJECTS FOR CLINICAL TRIALS, naming Gunther Winkler, Kathryn T. Hall, and Ted J. Kaptchuk. The entire content of the foregoing applications are incorporated herein by reference, including all text, tables and drawings.

### FIELD OF THE INVENTION

Technology provided herein relates, in part, to methods of conducting placebo-controlled treatment studies, method of normalizing, reducing or eliminating placebo effects and methods of detecting COMT polymorphisms.

### BACKGROUND OF THE INVENTION

Placebo effects are ubiquitous in clinical care and present both opportunities and significant issues. Opportunities exist for caregivers to enhance therapeutic effects of medications and medical procedures through exploitation of a patient's capability of a healing placebo response. It has been demonstrated that clinical intervention paired with placebo responses can significantly improve outcomes for patients.

In addition, placebo effects significantly impact the success of the development of new therapeutic interventions. Increasingly, many drugs, medical devices and clinical procedures that have considerable evidence and early demonstration of efficacy are not further developed because of the prohibitive cost of demonstrating the FDA required superiority to placebo controls in large clinical trials. This is one critical factor that has led to a slowing time to registration and has led to sharply rising costs of medicines and medical devices. Methods for reducing the size and cost of the required clinical trials and still demonstrating superiority to placebo is desired and has been sought after by the US government and especially by the FDA which has established a “Critical Path Initiative” to explore faster and cheaper development of new drugs, devices and procedures. Methods to reduce and control placebo effects during clinical trials are a critical element in achieving this goal. It has been known that some patients have a natural propensity to increased placebo response and it is desirable to identify and to exclude such patients from clinical trials. However, until now, methods to identify such patients prior to enrollment into clinical trials have failed or have provided inconsistent and unpredictable results. Establishing a systematic and accurate way to identify placebo responders and thereby predicting or even controlling placebo effects in clinical trials would be a major step towards more efficient, faster and cheaper drug development which would greatly benefit health care developers, patients and society in general.

### SUMMARY OF THE INVENTION

In some embodiments, the present invention is directed, in part, to methods and kits for identifying and/or randomizing subjects participating in clinical trials that may exhibit a placebo response and identifying treatments for subjects with varying degrees of placebo responses. In one aspect, a method of selecting subjects to participate in a clinical trial is disclosed. In another aspect, methods for treating a subject and determining a treatment dosage are disclosed. In yet another aspect, a protein or peptide assay for determining a catechol-O-methyltransferase (COMT) polymorphism is disclosed.

In certain aspects presented herein is a method of selecting a sub-group of human subjects comprising detecting the presence or absence of a catechol-O-methyltransferase polymorphism in a sup-population of human subjects and selecting a sub-group of human subjects from the sup-population wherein the sub-group comprises the presence of the catechol-O-methyltransferase polymorphism. In some aspects the method further comprises distributing the human subjects of the sub-population into at least two study groups and administering a placebo treatment to one or more of the study groups. In certain aspects the method comprises distributing human subjects that comprise the presence of one or more catechol-O-methyltransferase polymorphisms evenly or about equally among two or more study groups. In certain aspects the method comprises excluding human subjects that comprise the presence of one or more catechol-O-methyltransferase polymorphisms from two or more study groups. The method sometimes comprises administering an experimental treatment to subjects of the study groups, wherein the experimental treatment is configured to treat a symptom, and/or evaluating the efficacy of the experimental treatment to reduce or alleviate a symptom. In some embodiments a symptom is a subjective symptom. In certain aspects, the sub-population of human subjects are candidates for a placebo-controlled clinical trial. In some embodiments the catechol-O-methyltransferase polymorphism comprises a single nucleotide polymorphism in a catechol-O-methyltransferase gene and in certain aspects the single nucleotide polymorphism comprises at least one of an rs4680, rs4818, rs6269 and rs4633 polymorphism.

In some embodiments a method of selecting subjects to participate in a clinical trial includes identifying subjects with a catechol-O-methyltransferase (COMT) polymorphism, where the COMT polymorphism modulates a placebo effect in the subjects, and selecting subjects to participate in the clinical trial based on their COMT polymorphism. The COMT polymorphism can be at least one of rs4680, rs4818, rs6269, and rs4633. In one embodiment, the COMT polymorphism is the COMT Val158met, also known as rs4680, the valine/methionine genotype, methionine/methionine genotype or valine/valine genotype. The COMT polymorphism may also indicate an increased likelihood the subject would exhibit a placebo effect, such as a high or low placebo effect when compared to subject that lack the COMT polymorphism. In some embodiments, subjects with a COMT polymorphism can be included or excluded from a clinical trial. In some other embodiments, subjects with the COMT polymorphism can be administered a modified or alternative treatment.

One aspect can include a method for determining a response to a treatment of a subject having, suspected of having, or at risk for developing a disorder, such as, irritable bowel syndrome, diabetes, autoimmune disorders, inflammation, neurological disorders, chronic and acute pain, cancer, cancer treatments, allergies, depression, migraines, addiction, obesity, cardiovascular disorder and other disorders, syndromes, or diseases. The method can also include subjects in a clinical trial. The method can include determining a catechol-O-methyltransferase (COMT) polymorphism in the subject, identifying a treatment for the subject based on the COMT polymorphism and administering the treatment to the subject. A sample, such as a blood, urine, ascites, cerebrospinal fluid, bronchial lavage, oral washings and sputum, Pap smears, tissue biopsies or organs, bile, fecal matter, or other bodily fluids, tissues or parts that may include nucleic acids can also be obtained from the subject and analyzed for the COMT polymorphism. Depending on the COMT polymorphism, a subject can be assessed for a likelihood of exhibiting a placebo effect, such as an increased or decreased likelihood of exhibiting the placebo effect. Identifying a subject that has an increased or decreased likelihood of exhibiting the placebo effect often includes detecting the presence or absence of a COMT polymorphism in the subject. In some embodiments subjects that are heterozygous for the presence of a COMT polymorphisms are about 1.1, 1.2, 1.3, 1.4, 1.5, 1.6, 1.7, 1.8, 1.9, 2.0, 2.1, 2.2, 2.3, 2.4, 2.5 fold or more likely to have a placebo response than subjects that are homozygous for the absence of the same COMT polymorphism. In some embodiments subjects that are homozygous for the presence of a COMT polymorphisms are about 1.1, 1.2, 1.3, 1.4, 1.5, 1.6, 1.7, 1.8, 1.9, 2.0, 2.1, 2.2, 2.3, 2.4, 2.5 fold or more, or 3-fold or more likely to have a placebo response than subjects that are homozygous for the absence of the same COMT polymorphism.

A method for treating a subject by determining a catechol-O-methyltransferase (COMT) polymorphism in the subject, identifying a treatment for the subject based on the COMT polymorphism and administering the treatment to the subject is also disclosed. The method can also include subjects in a clinical trial. Furthermore, the method can be directed to treating a subject with, suspected of having, or at risk for developing a disorder, such as cardiovascular disorder, irritable bowel syndrome, diabetes, autoimmune disorders, inflammation, neurological disorders, chronic pain, cancer, cancer treatments, allergies, depression, migraines, addiction, obesity, and other disorders, syndromes, or diseases. The method can also include obtaining a sample from the subject and analyzing the sample for the COMT polymorphism. The subject can have an increased likelihood of exhibiting a placebo response.

In one embodiment, the identified treatment can include modulating the treatment for the subject with the COMT polymorphism and administering the modulated treatment. In another embodiment, the identified treatment can be an alternative treatment for the subject with the COMT polymorphism, and that treatment can be administered to the subject. The modulated or alternative treatment can include adjusting a dosage of the treatment administered to the subject depending on the COMT polymorphism.

A treatment dosage can also be determined by determining a catechol-O-methyltransferase (COMT) polymorphism in a subject and modulating the treatment dosage administered to the subject based on the COMT polymorphism. The subject may also have, be suspected of having, or at risk for developing a disorder, such as cardiovascular disorder, irritable bowel syndrome, diabetes, autoimmune disorders, inflammation, neurological disorders, chronic pain, cancer, cancer treatments, allergies, depression, migraines, addiction, obesity, and other disorders, syndromes, or diseases. A sample, such as a blood, urine, ascites, cerebrospinal fluid, bronchial lavage, oral washings and sputum, Pap smears, tissue biopsies or organs, bile, fecal matter, or other bodily fluids, tissues or parts that may include nucleic acids can also be obtained from the subject and analyzed for the COMT polymorphism. The treatment dosage administered can be modulated, increased or decreased, or an alternative treatment can be identified for the subject with the COMT polymorphism.

Another aspect includes kits and assays for determining a catechol-O-methyltransferase (COMT) polymorphism in a subject. A protein or peptide assay can include at least one antibody specific for the COMT polymorphism, reagents for measuring catechol-O-methyltransferase enzymatic activity, and/or reagents for analyzing catechol-O-methyltransferase protein.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A is a bar graph showing the effect of the rs4680 COMT genotype on change in IBS-SSS after receiving a placebo treatment. Number of Val158met met alleles showed a significant linear effect on IBS-SSS (beta=0.17; p=0.032). IBS-SSS includes abdominal pain severity, abdominal pain frequency, abdominal distention severity, dissatisfaction with bowel habits, and disruption of quality of life. Change in IBSSSS=(IBS-SSS at baseline—IBS-SSS at 3-weeks). Regression model included COMT genotype (number of met alleles) and baseline IBS-SSS. Error bars indicate the standard error of the mean. N=104.

FIG. 1B is a bar graph showing the effect of the rs4633 COMT genotype on change in IBS-SSS after receiving a placebo treatment.

FIG. 1C is a bar graph showing the effect of the rs4818 COMT genotype on change in IBS-SSS after receiving a placebo treatment.

FIG. 2A is a bar graph showing the interaction effect of rs4680 COMT genotype and placebo treatment arms on change in IBS-SSS. The interaction between COMT genotype (number of met alleles) and treatment arm was statistically significant (beta=0.17; p=0.035). Regression model included the following parameters: COMT genotype (number of met alleles), treatment arm, baseline IBS-SSS and their interaction (COMT genotype x treatment arm). Error bars indicate the standard error of the mean. N=104.

FIG. 2B is a bar graph showing the interaction effect of rs4633 COMT genotype and placebo treatment arms on change in IBS-SSS.

FIG. 2C is a bar graph showing the interaction effect of rs4818 COMT genotype and placebo treatment arms on change in IBS-SSS.

FIG. 3A is a bar graph showing the interaction effect of rs4680 COMT genotype and placebo treatment arms on Adequate Relief. The interaction (COMT genotype x treatment arm) was statistically significant (beta=0.25; p=0.009), but not COMT genotype (beta=0.01; p=0.954). Adequate Relief was assessed by a single dichotomous categorization at three weeks, which asked patients: “Over the past week have you had adequate relief of your IBS symptoms?” Regression model parameters included: COMT genotype (number of met alleles), treatment arm and their interaction (COMT genotype x treatment arm). Error bars are standard error of the mean. N=102.

FIG. 3B is a bar graph showing the interaction effect of rs4633 COMT genotype and placebo treatment arms on Adequate Relief.

FIG. 3C is a bar graph showing the interaction effect of rs4818 COMT genotype and placebo treatment arms on Adequate Relief.

FIG. 4 shows results of a placebo study testing the placebo response of subjects to a pain stimulus. Each bar represents a quantitation of placebo effect (y-axis) of subjects comprising a Val/Val genotype (Val/Val), Val/Met genotype (Val/Met) or a Met/Met genotype (Met/Met) for amino acid 158 of COMT (COMT Val158met). N=48. (p=0.058) The study shown was conducted at Mass General Hospital, Harvard Medical School.

### DETAILED DESCRIPTION OF THE INVENTION

The present invention is directed to the association of the COMT polymorphisms (e.g., rs4680 (Val158met)) with placebo response/effect modulation. The present invention is also directed to the detection of COMT polymorphisms and clinical trial management. The modulation of placebo response or effect conferred by COMT polymorphisms may have implications for personalized medicine and development of strategies in clinical trials and treatment regimens.

Certain exemplary embodiments will now be described to provide an overall understanding of the principles of the structure, function, manufacture, and use of the compositions and methods disclosed herein. One or more examples of these embodiments are illustrated in the accompanying drawings. Those skilled in the art will understand that the compositions and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Subjects

A subject can be any living or non-living organism, including but not limited to a human, non-human animal, plant, bacterium, fungus, virus or protist. A subject is sometimes a human subject. A subject may be any age (e.g., an embryo, a fetus, infant, child, adult). A subject can be of any sex (e.g., male, female, or combination thereof). A subject may be pregnant. A subject can be a patient (e.g. a human patient).

In some embodiments a subject has or is suspected of having a disorder, ailment, disease or condition. In some embodiments a condition includes, for example, damage or injury (past or present), psychological conditions, genetic conditions, chronic or acute conditions, and the like or combinations thereof. A disorder can be a genetic disorder (e.g., a disorder, ailment or disease associated with a genetic variation in a subjects genome). A subject can be a subject with a predisposition (e.g., a genetic predisposition) towards developing a disorder. A subject may be suspected of having a disorder due to a genetic predisposition of having said disorder. In some embodiments, a subject comprises a genetic variation and is determined to have a disorder due to the presence of the genetic variation.

Subjects further include those that are at increased risk of developing a disorder or disease. “At risk” subjects typically can have risk factors for developing or having a particular disorder or disease. Non-limiting examples of risk factors include a family history (e.g., genetic predisposition), the presence or absence of a genetic variation, gender, race, age, lifestyle (e.g., diet, smoking), occupation, environmental factors (e.g., exposure), the like or combinations thereof.

In some embodiments a subject is a subject in need of a treatment (e.g., a medical treatment, an experimental treatment) or drug (e.g., pharmaceutical drug, experimental drug). In some embodiments a subject in need of a treatment or drug is a subject having or suspected of having a disorder, disease or condition. In some embodiments a subject in need of a treatment or drug is a subject experiencing one or more symptoms of a disorder, disease or condition. In some embodiments a subject in need of a treatment or drug is a subject at risk of developing a disorder or disease. In some embodiments a subject in need of a treatment or drug is, or is suspected of being, infected with a pathogen.

In certain embodiments a subject having, suspected of having or at risk of having a disorder, ailment, disease or condition is considered for participating in a clinical trial or study group. In certain embodiments a subject having, suspected of having or at risk of having a disorder, ailment, disease or condition is a candidate for participating in a clinical trial or a candidate for receiving an experimental treatment. In certain embodiments a subject having, suspected of having or at risk of having a disorder, ailment, disease or condition is selected to participate in a clinical trial by a method described herein. In certain embodiments a subject having, suspected of having or at risk of having a disorder, ailment, disease or condition is selected to receive an experimental treatment by a method described herein. In certain embodiments a subject having, suspected of having or at risk of having a disorder, ailment, disease or condition is selected to receive a placebo treatment by a method described herein.

Symptoms

In some embodiments a subject is experiencing one or more symptoms. Symptoms can be chronic or acute. A symptom can be constant or intermittent. In certain embodiments a symptom can be the absence of a normal sensation or biological process. A symptom can be caused by any disorder, disease or condition. In some embodiments a cause of a symptom is not known. In some embodiments a symptom is related to a psychological disorder. In some embodiments a symptom is manifested by a psychological disorder. In some embodiments a symptom is a neurological symptom or related to a neurological disorder, disease or condition.

In certain embodiments a symptom can be any measureable and/or observable symptom (e.g., a symptom that can be measured and/or observed by a medical professional). Non-limiting examples of measurable and/or observable symptoms include inflammation, swelling, infection, organ failure, hyperplasia, the presence of cancer (e.g., a tumor, lymphoma), coughing, wheezing, temperature (e.g., fever, hypothermia, hyperthermia), the presence of a pathogen, rash, weight gain, jaundice, bruising, convulsions, seizures, discharge, bleeding, sweating, tremors, urinary incontinence, bowel obstruction, arrhythmia, the like or combinations thereof.

In certain embodiments a symptom is a subjective symptom. A subjective symptom can be a subject's perception of any physical or mental symptom. In some embodiments a subjective symptom is a symptom experienced by a subject where the presence, absence, description and/or degree (e.g., amount, intensity) of the symptom is determined according to a subjective response from the subject experiencing the symptom. In some embodiments the presence, absence, description and/or degree of a subjective symptom can only be assessed by a subjective response. A subjective response can include any suitable audible and/or physical response that communicates the presence, absence, description and/or degree of a symptom to a another (e.g., a third party, a medical professional). Non-limiting examples of audible responses include one or more sounds, gestures, verbal responses and/or spoken words. Non-limiting examples of a physical response include gestures, signs (e.g., use of sign language), physical movements (e.g., any suitable movement of a body part, twitching, reflex movements, the like, or combinations thereof), drawing, writing (e.g., written words, written responses), the like or combinations thereof. A response can be communicated directly or indirectly to another. For example, a response can be communicated indirectly to another by means of a communication device (e.g., a computer, cell phone, an app (e.g., a health tracking app)) and the like).

A subjective response is often a response to a suitable sensory stimuli configured to induce a response. In some embodiments a sensory stimuli is a query or question directed to a subject. Often a sensory stimuli is a question asking a subject about the presence, absence or degree of a symptom. In some embodiments a sensory stimuli is a question asking a subject to describe a symptom. In certain embodiments a sensory stimuli is a question asking a subject about the presence, absence or degree of a stimuli. In certain embodiments a sensory stimuli is a question asking a subject about the presence, absence or degree of a symptom during or following a treatment. In some embodiments a subjective response is a subject's answer to a question regarding a symptom (e.g., regarding the presence, absence, description and/or degree of a symptom). In some embodiments a sensory stimuli is a visual stimuli. Non-limiting examples of visual stimuli include written questions, pictures, light, color, shades, shapes, the like, changes thereof, or combinations thereof. In some embodiments a sensory stimuli is a sound (e.g., music), smell, and/or taste (a food or drink). In some embodiments a sensory stimuli is an epidermal stimuli, non-limiting examples of which include a suitable touch (e.g., grasp, hold, poke, prick, brush) wind, heat, cold, electrical stimuli, the like or combinations thereof. In some embodiments a subjective response is not in response to a stimuli configured to induce a response. For example, sometimes a subject provides a voluntary subjective response regarding a symptom without a stimuli or provocation.

Non-limiting examples of a subjective symptom include any pain, (e.g., phantom pain, headache, muscle pain, aches, abdominal (stomach) pain, chest pain, heart burn, and the like), discomfort, pressure, cramping, sensations of constipation, sensations of bloating, nausea, feeling sick, tingling, sensations of touch, sensations of warmth, heat, hot flashes, burning, cold, freezing, weakness, fatigue, anxiety, restlessness, restless leg syndrome, vestibular sensations (e.g., dizziness, balance, spatial orientation), inability to concentrate, memory problems, sensations of sight/vision, blurred vision or blindness (e.g., without a biological, biochemical or physiological explanation), anger, cravings, disrupted mood, optical flashes or pulses of light, perception of color, light, and/or darkness, hallucinations, insomnia, hearing and auditory sensations, tinnitus, sensations of smell, the like, presence thereof, lack thereof, or combinations thereof.

COMT

Catecholamines play a key role in cognitive, behavioral, sensory, endocrine and autonomic nervous system regulation. Thus functional polymorphisms in catechol-O-methyltransferase (COMT, EC2.1.1.6, UniProtKB: P21964), an enzyme that metabolizes catecholamines is associated with a variety of clinical conditions. Catechol-O-methyltransferase (COMT) is an enzyme that catalyzes the O-methylation of various compounds, like catechol estrogens and dietary polyphenols, using S-adenosylmethionine (SAM) as the methyl donor and has also a role in dopamine inactivation,

COMT is encoded on human chromosome 22 (Entrez Gene ID: 1312, NCBI Reference Sequence: NG_{—}011526.1 showing nucleotides 1 to 35236 of human COMT gene) and several polymorphisms in the gene can affect the expression and function of the enzyme. As described herein, the presence of a polymorphism (e.g., a particular genotype of a polymorphism) in a COMT gene can be associated with a placebo response and/or an increased likelihood that a subject will have a placebo response. A polymorphism can comprise one or more single nucleotide polymorphisms (SNP). In some embodiments a COMT comprises 1 to 20, 1 to 10, or 1 to 5 polymorphisms. In some embodiments a COMT comprises 1, 2, 3, 4, 5, 6, 7, 8, 9 or 10 polymorphisms some or all of which can be associated with a placebo effect and/or an increased likelihood that a subject will have a placebo response. A polymorphism can be in a COMT gene, a transcribed region of a COMT gene, a COMT intron, a COMT exon, a COMT coding sequence, a regulatory regions that can effect COMT expression (e.g., mRNA expression or protein expression), the like or combinations thereof. In some embodiments the presence of a particular polymorphism in a COMT nucleic acid results in an amino acid mutation (e.g., an amino acid substitution, insertion or deletion). In some embodiments an amino acid mutation in a COMT polypeptide reduces COMT enzyme activity compared to a wild-type (e.g., non-mutated enzyme) by 2-fold or more, 3-fold or more, 4-fold or more, 5-fold or more or 6-fold or more. In certain embodiments reduced COMT activity is associated with a placebo effect and/or an increased likelihood that a subject will have a placebo effect. In some embodiments the presence of a COMT polymorphism (e.g., a particular genotype of a COMT polymorphism) results in the reduced expression of a COMT polypeptide. In some embodiments the presence of a COMT polymorphism reduces expression of a COMT polypeptide compared to expression of a COMT polypeptide from a wild-type gene (e.g., a majority genotype, the absence of a COMT polymorphism, a COMT gene lacking said polymorphism) by 2-fold or more, 3-fold or more, 4-fold or more, 5-fold or more or 10-fold or more. In certain embodiments the presence of a polymorphism in a COMT nucleic acid that reduces expression of a COMT polypeptide is associated with a placebo effect and/or an increased likelihood that a subject will have a placebo effect. In some embodiments a method comprises selecting a sub-group of human subjects wherein the presence of a COMT polymorphism is detected in subjects of the sub-group and the presence of the COMT polymorphism results in reduced activity or reduced expression of a COMT polypeptide.

The presence of a polymorphism refers to the presence of a specific allele of a polymorphism. The presence of a specific allele can refer to a heterozygous or homozygous genotype comprising the allele. A detected polymorphism in a COMT gene can be present in one or both alleles (e.g., both alleles of a genotype for a diploid subject). In some embodiments a detected COMT polymorphism is present in one allele and the subject is heterozygous for the COMT polymorphism. In certain embodiments a detected COMT polymorphism is present in both alleles and the subject is homozygous for the COMT polymorphism. In some embodiments the presence of a COMT polymorphism refers to the presence of a recessive allele of a polymorphism. In some embodiments the presence of a COMT polymorphism refers to the presence of a minor allele of a polymorphism. In some embodiments the presence of a COMT polymorphism indicates the presence of a specific genotype in a subject. A genotype can include one or more than one COMT polymorphism. For example the presence of a COMT polymorphism can include the presence of genotype that includes an rs4680 polymorphism and an 4618 polymorphism.

The COMT Val158met or rs4680 polymorphism is a G to A transition at codon 158 (e.g., amino acid 158, P21964) in the transmembrane form and codon 108 (e.g., amino acid 108) in the secreted form of the COMT protein. The COMT rs4680 polymorphism occurs at nucleotide number 973 of the COMT mRNA transcript variant X1 shown herein. The rs numbers (“rs#”; “refSNP cluster ID number”) shown herein represent a well-known nomenclature for single nucleotide polymorphisms (SNP). The reference rs numbers and information regarding their representative SNPs (e.g., nucleotide positions) can be searched and found online at URL http://www.ncbi.nlm.nih.gov/SNP/, accessed Oct. 13, 2014, for example. The G allele of rs4680 codes for a valine and is sometimes referred to herein as the Val158 allele. The A allele of rs4680 codes for methionine and is sometimes referred to herein as the Met158 allele. The presence of an rs4680 polymorphism often refers to the presence of an A allele of the rs4680 polymorphism. The presence of an A allele can refer to a heterozygous or homozygous genotype comprising an A allele of an rs4680 polymorphism. In some embodiments, detection of the presence of the A allele of rs4680 (Met158) determines the presence of an rs4680 polymorphism. In some embodiments, detection of the absence of an rs4680 refers to detecting the absence of an A allele of an rs4680 polymorphism. In some embodiments, detection of the presence of two G alleles of an rs4680 (Val158)(e.g., a G/G homozygote) determines the absence of an rs4680 polymorphism. The presence or absence of an rs4680 polymorphism can be determined by a suitable method or by a method described herein. The Val158 form of a COMT polypeptide is 3 to 4-fold more active than the Met158 form at body temperature. As a result of this mutation, homozygotes for the Val158 allele, in the absence of substances that block the enzyme, have been shown to have significantly lower levels of catecholamines e.g. dopamine and epinephrine as compared to Met158 allele homozygotes. This functional polymorphism has been correlated with variations in memory function, cognition, attentional processing, high blood pressure, pain processing and sensitivity.

Other polymorphisms in COMT including rs4818 and rs4633 have been shown to affect the stability of the COMT messenger RNA and thus the expression levels of the enzyme. The rs4633 polymorphism represents a C to T transversion. The presence of an rs4633 polymorphism is indicated by the presence of the T allele. The presence of an rs4818 polymorphism is indicated by the presence of the C allele. The rs6269 polymorphism represents a A to G transversion. The presence of an rs6269 polymorphism is indicated by the presence of the G allele.

Detecting the presence of a COMT polymorphism can include detecting the presence of one or more COMT polymorphisms. In certain embodiments detecting the presence of a COMT polymorphism comprises detecting the presence and/or absence of an rs4680 polymorphism, an rs4818 polymorphism, an rs6269 polymorphism, and an rs4633 polymorphism, alone or in combination. For example, a subjects genotype can comprise the presence of an rs4680 polymorphism, the presence of an rs4818 polymorphism and the absence of an rs4633 polymorphism.

The data presented in the Examples section herein suggests that genetic variations that change the expression or activity of COMT can affect a subject's placebo response. For example, a significant correlation of the rs4680 COMT polymorphism and the resulting response to a placebo treatment or a placebo inducing therapeutic encounter between a subject and a caregiver is demonstrated herein. In some embodiments, a therapeutic encounter with a caregiver is considered an active component of a non-specific placebo treatment. For example in some embodiments the presence of a therapeutic encounter is used to identify reliable predictors of a placebo effect. Other studies did not include an arm in which a placebo treatment was administered within the context of an augmented therapeutic encounter. Indeed, many of these studies were done with volunteer subjects in experimental rather than clinical contexts in which there was no therapeutic encounter at all.

Some skilled in the art believe it is unlikely that a single gene locus (e.g., like a polymorphism in COMT) can fully account for a complex behavioral phenotype such as a placebo response. The data presented suggests that a single nucleotide polymorphism in COMT (e.g., like rs4680) can account for a significant increase in a subjects placebo response. Therefore, the data presented herein was surprising and unexpected.

In some embodiments, a COMT polymorphism is at least an rs4680, rs4818, rs6269, and/or rs4633 polymorphism. A COMT polymorphism can be an rs4680 polymorphism and encode a methionine/methionine haplotype, a valine/methionine haplotype, or a valine/valine haplotype. Some examples of COMT polymorphisms, but not limited to the listed COMT polymorphisms, that can be used with the methods disclosed herein are show in Table 1.

TABLE 1
COMT Polymorphisms

| COMT | Context Sequence |
|---|---|
| rs1055503 | AAGGGAGGGGAGGGGGCCCCGACGG[A/G]GCCACAGTA |
| | AAGGAGTGGAGGGGCC |
| rs1109436 | TGCAATTCAAAATCAAGGGCTGCTT[C/T]GAGGAGGCC |
| | TCTCCACCGGGCTGCT |
| rs11544670 | TGCCGGAGGCCCCGCCTCTGCTGTT[G/T]GCAGCTGTG |
| | TTGCTGGGCCTGGTGC |
| rs11569716 | TCCGTGTCTGCTTCTGTATTTTGTG[C/T]GGTTTTAGA |
| | GGATCCCTGGGCTGCC |
| rs12814 | CCCGGCCCCCCTCTCGGGCTCTCTC[A/T]CCCAGCCTG |
| | GTACTGAAGGTGCCAG |
| rs13055002 | TGGCCCCAGGGGCTAGGCACAGGCG[C/T]GGTGCCGTG |
| | GCCTAGTGAGGAGCAC |
| rs13306281 | CGAGCAGAAGGAGTGGGCCATGAAC[A/G]TGGGCGACA |
| | AGAAAGGTGGGGTCCG |
| rs1544325 | GAGACAAATTAGAAATGTCAGTCTG[A/G]AGAGAGTGG |
| | TAGGTAGCCAGATACT |
| rs165599 | TGTTAGCCCCATGGGGACGACTGCC[A/G]GCCTGGGAA |
| | ACGAAGAGGAGTCAGC |
| rs165631 | ACCGGTACCTGCCGGACACGCTTCT[C/T]TTGGAGGTG |
| | AGCCCCAACCAGGATG |
| rs165722 | CTTCCCTGTTCTCTTCTGCTCTGTC[T/C]TCTGGTGCC |
| | CTGAGGCTGGCCTCCA |
| rs165728 | AGTGAGGATGCAGTGCTGGTTTCTG[T/C]CCACCTACA |
| | CCTAGAGCTGTCCCCA |
| rs165737 | TTGGGACACCCACCCTCACGGCCTC[C/T]CCACCTGGT |
| | GCTCGCTCACCTGCAG |
| rs165774 | AAACTGGACACTGCTGTTAGCAGCC[A/G]GACTAGGAG |
| | CACGAGGGGCACAGCC |
| rs174675 | GAAATGCTGTCAGAAGCCTACCCCA[C/T]GGTACCTGT |
| | CATGGGCCTTTTCATT |
| rs174695 | GGGACAGGCGGGCACTGGGTGCCTC[C/T]TTGCACCAG |
| | CCAGGCCCAGCCTGCA |
| rs174696 | CTGCGTCCGGCCGTATTCCAGCTTT[C/T]AAAACAACA |
| | AAAAACAACAAAAACT |
| rs174697 | GGGTTCTGTGAGCATCGGAGGCACG[A/G]GGGGTGAGG |
| | GGCTCAGGAGCAGGTT |
| rs174699 | CTCCTCTGACACTGTCGCTTCTCCA[T/C]GGCATTAGA |
| | TTTTCAGTCCTGCTCA |
| rs2239393 | TTACAGGAGAAGCTGTTATCACCCC[A/G]TTTCCAGGG |
| | GGCTGGGAACCCTGGG |
| rs28562050 | GAGGTTTCTCCCGATAGTGGACCCC[A/C]CGGGCCATG |
| | CCCTATGGCTGCGTGT |
| rs28565095 | TGGGGAGCCAGCTGCCTGTGCAGGT[A/G]CAAAATGGG |
| | TGGCAGAAGTGGGGTG |
| rs28621910 | CCATGCAGGTCTGGCCCATGGAAGG[C/G]AGGGGAGGG |
| | GGCCCCGACGGGGCCA |
| rs2904564 | CGGACTAGGAGCACGAGGGGCACAG[C/G]CCCCATGCC |
| | TGGCTAGGTAGGGCCG |
| rs3218737 | CGTGGGCGACAAGAAAGGTGGGGTC[C/T]GGGCCAGCA |
| | GGTGCTCAGCTCTGGG |
| rs34068292 | AGCCACTGCTCGTGCAAAGAAAGCA[C/T]GTGTCTCCT |
| | GCAGATCTTCCTCCTG |
| rs34234839 | TCCTCTGGAGTCCCAGGGTGCCAGG[G/T]TCCCTGATG |
| | ACCCCTGCAGGCCCTG |
| rs34300220 | CTGGGCCCAAGCAATCCTCCCACTT[C/T]AGCCTCCTG |
| | AGTAGCTGCAGCTATA |
| rs34446512 | GCTCTGGGCCCAGCCTGCTCTCCCC[C/G]AAGCAAGCC |
| | ACTGCTCGTGCAAAGA |
| rs34450201 | GCCTTCTGCTGTCTCTTCTGAGGCC[A/G]GAAAGGCGA |
| | AGCACTGCCCTCGCCC |
| rs34691071 | GAGCTGGAGGGGGGGTCTTCCTGGG[C/T]GGCCTGTGT |
| | AGCAGCAGCTCCAGGC |
| rs34819182 | CTCTGGGACAGGGACCCAGGACCAG[G/T]CATCAAAGC |
| | CCTTACAGGAGAAGCT |
| rs34966851 | ATCTGGATTTGATGGCATTTTTATT[A/G]TGGTGTTGT |
| | TTTAGAGACAGTGTCT |
| rs35478083 | GCAGCACCAGCCCCTCTGGGCCCCA[C/T]GTGGCACAG |
| | AGTGGAAGCATCTCCT |
| rs35481270 | AGAGGCAATGGCTCAGACCAGCTCC[C/T]GCATCCCTG |
| | TAGTTGCCTCCCTGGC |
| rs35593000 | TTGCTTACAGAAGAGGCAATGGCTC[A/C]GACCAGCTC |
| | CCGCATCCCTGTAGTT |
| rs35617967 | GGGCTCTGGCCTTTGGCCTTTTCCT[C/G]TGAGCTGCA |
| | TGTTGGCCACTGTGAC |
| rs35777291 | CTGAAAACATCTTGTAAGCTTTTTT[A/T]GGCCAATAT |
| | ATTATCAAACGCAATT |
| rs35919169 | GGACCTTGGTCATCGCCAGGTTAGG[A/G]TTTATGTTG |
| | ATGATTATCCAAAGGC |
| rs36082074 | CTGGGGACAGGTGGACCCCGCAGCA[A/G]CACCAGCCC |
| | CTCTGGGCCCCATGTG |
| rs3788319 | GTGAAGTGATCTGACGTTGGGTGGG[A/G]GTCTCCGGA |
| | CTTGGGGTGGGGAATC |
| rs3788320 | GAGGGCCTTGGTGACTTCTCCAACA[A/G]GCTCCCTGT |
| | TGGAGGCCTTGGGGTA |
| rs3810595 | TACAGCCTCTCCTTTGGCCACCCGT[C/G]ACTACCCCC |
| | AACTCCGGGCCATGGG |
| rs45454096 | ACTGGGATAGGGTGTGGGGAATTCG[A/G]ACCGCTGTG |
| | AAGTGATCTGACGTTG |
| rs4633 | CCAAGGAGCAGCGCATCCTGAACCA[C/T]GTGCTGCAG |
| | CATGCGGAGCCCGGGA |
| rs4646312 | TGGTTTGTGTATGTTCTTGGTAAAC[T/C]AGCCCTTGG |
| | TCTTACACATCATTTC |
| rs4646315 | TTCAGTCAGCCTCAGCCTCTCCAAA[C/G]AGCCAGGCA |
| | TTCCAGTAGAGCCCTG |
| rs4646316 | CCCAGACCAGACACCAGGGCAGAAA[C/T]GGCACAGGA |
| | CCAAGGAGATGGGGTG |
| rs4680 | CCAGCGGATGGTGGATTTCGCTGGC[A/G]TGAAGGACA |
| | AGGTGTGCATGCCTGA |
| rs4818 | GCCTGCTGTCACCAGGGGCGAGGCT[C/G]ATCACCATC |
| | GAGATCAACCCCGACT |
| rs4986871 | ACCATCGAGATCAACCCCGACTGTG[C/T]CGCCATCAC |
| | CCAGCGGATGGTGGAT |
| rs5992500 | TGCCATGGTCGGATGAGAGCAGCCA[C/T]GGACAATGT |
| | CTGTGCAATTGTGCGT |
| rs5993882 | AAAGTTACGCTTAATAATGAATGTT[T/G]CAGCACTTT |
| | CTTCTCTTCAGGTATT |
| rs5993883 | GTTGAAAGTTACTGAAAACATCTTG[G/T]AAGCTTTTT |
| | TAGGCCAATATATTAT |
| rs5993889 | GTAAGCAAACCCTTTTCTGATAGCA[G/T]AGATAGGCA |
| | AGCATCCTATGAGGTT |
| rs61910731 | TCCCCAACCCTGCACAGGCAAGATC[G/T]TGGACGCCG |
| | TGATTCAGGAGCACCA |
| rs6267 | GCTGCAGCATGCGGAGCCCGGGAAC[G/T]CACAGAGCG |
| | TGCTGGAGGCCATTGA |
| rs6268 | GCACCAGAGGGCACGAGAAGGCTGG[C/T]TCCCTGGCG |
| | CTGACACGTCAGGCAA |
| rs6269 | GCATTTCTGAACCTTGCCCCTCTGC[G/A]AACACAAGG |
| | GGGCGATGGTGGCACT |
| rs6270 | CTGAGGCACTGGGGCTGGGGCCTGT[G/C]CCTTATCGG |
| | CTGGAACGAGTTCATC |
| rs72549343 | TCCTGCAGCCCATCCACAACCTGCT[—/A]CATGGGTGA |
| | CACCAAGGAGCAGCGC |
| rs737864 | GTTGCTTTTTATTGTTTAAAGTGAC[C/T]CTCCAAGCC |
| | TGAGGGACCGTAGGAG |
| rs737865 | GCTTTTTGGATTTTTCCAGCCAGGG[A/G]TTTTTGTGT |
| | CCTGTTGCTTTTTATT |
| rs737866 | GCTAACAGACCTGCTTTTTGGATTT[C/T]TCCAGCCAG |
| | GGATTTTTGTGTCCTG |
| rs739368 | ACTGTGTGCTCCGGTTGTTGCATCC[A/G]AGGGTTGAT |
| | CGGATGGTGGTTCCCA |
| rs740601 | CAGGGCCCCAAGGTGGGCGGTTCGG[G/T]GATTCAGAG |
| | AGGGCAGCTCTGTGTT |
| rs740602 | AGCATGCGGAGCCCGGGAACGCACA[A/G]AGCGTGCTG |
| | GAGGCCATTGACACCT |
| rs740603 | TGTGAGGCACTGAGGATGCCCTCAC[G/A]CGTGCATCT |
| | GCATGTGGCGTGCATG |
| rs769224 | ACCACTGGAAGGACCGGTACCTGCC[A/G]GACACGCTT |
| | CTCTTGGAGGTGAGCC |
| rs9306235 | GCCCATCCCAGAGGCATGTGGGGTC[A/G]GATACCAGT |
| | GTTTCAAGGCACCTGC |
| rs9332360 | AAATCCCCTAGAAGCCTGGTGTCCG[C/T]ATGACCTCC |
| | CCCTAGGGCGGAGCCT |
| rs9332367 | GGTAGGGCCGCTGGACCCTGGACAC[A/G]GATTGGAAG |
| | GAACCAGCACTAGCAG |
| rs9332377 | AACCCCTCTCCTTGGGTGCCTCTCC[C/T]TCATAGGCC |
| | TGAGTTCCTGGCACTG |
| rs9332380 | CCTGACATGCTAACCTCTCTGAACT[A/G]CAACACTGG |
| | ATTGTTCTTTTTTAAG |
| rs933271 | GTGGTTACTTTCTGGAGAGAGCATG[T/C]GGCATGCAG |
| | GAGCTGGAGGGGGGGT |

The most extensively studied COMT single nucleotide polymorphism (SNP), rs4680 or Val158met, is a G to A transition that encodes a valine (Val) to methionine (Met) substitution at amino acid 158 in the membrane form of the enzyme. The Val variant is 3-4 times more enzymatically active than the Met variant. The differences in enzymatic activity are inversely correlated to endogenous levels of dopamine and other COMT substrates including epinephrine, norepinephrine and catechol estrogens. A second COMT SNP, rs4818, encodes a synonymous C to G transversion in the same exon as rs4680 and the two SNPs are in partial linkage disequilibrium. The presence of an rs4818 polymorphism is indicated by the presence of the C allele of rs4818. Rs4818 has been associated with differential stability of COMT mRNA secondary structure as well as a series of clinical outcomes some of which are shared with rs4680 associations.

### Placebo Effects

In some embodiments placebo effects include a patient's response to inert substances, such as sugar pills, as well as the therapeutic context that surrounds medical treatment. The therapeutic encounter can include a mixture of the symbols and rituals of health care, combined with the charged emotional reactions that arise when patients encounter healers, including trust, empathy, hope, fear, trepidation, and uncertainty. In certain embodiments, by using sugar pills, saline injections, or even sham surgery, placebo research isolates medicine's provision of care from the direct effects of genuine medications or procedures. In some embodiments a clinical encounter alone—without the provision of any therapeutic treatment—can alleviate pain, improve sleep, relieve depression, and improve the symptoms of a wide variety of illnesses, including irritable bowel syndrome, benign prostatic hypertrophy, asthma, Parkinson's disease, heart disease, and migraine. In some embodiments placebos can help patients experience less fatigue, nausea, pain, and anxiety that are associated with cancer. Placebo treatment may also promote more healthful behaviors. In some embodiments, placebos can behave like drugs and the placebo effect can sometimes make drugs more effective. In some embodiments the paraphernalia of care (pills, needles, etc.) and the patient-provider relationship can be added incrementally in a manner analogous to dose dependence (the higher the dose, the greater the effect). In some embodiments a placebo effect can boost the efficacy of many powerful medications. For example, when morphine is given by injection in full view of the patient, it is almost twice as effective as when it is given through an intravenous line without the patient knowing it is being administered.

A placebo effect can be variable in its magnitude and reliability both with subjectively, patient-reported symptoms and with objectively measured symptoms. As disclosed in the Examples, variations in the COMT (catechol-O-methyltransferase) gene are correlated to placebo effects among patients with irritable bowel syndrome participating in clinical trials. A gene often included introns, exons, translated and untranslated regions. In some embodiments a gene includes gene expression regulatory regions, non-limiting examples of which include promoters, enhancers, methylated or unmethylated nucleotides, TATA box, poly A regions, regions involved in RNA stability, the like or combinations thereof. In some embodiments a gene includes translational regulatory regions non-limiting examples of which include ribosome binding sites, initiation factor binding sites, Kozak sequences, 5′-cap, ATG, CTG, stop codons, hairpin loop, palindromic sequences, regions involved in RNA stability, the like or combinations thereof. Patients with a genotype of Met/Met (e.g., having two copies of the rs4680 methionine allele) were shown to be more likely to respond to a placebo treatment, while the genotype of Val/Val (e.g., having two copies of the valine allele) responded the least. The response of patients with one copy each of methionine and valine fell in the middle. Accumulation of higher concentrations of catecholamines, such as dopamine, in patients with the Met/Met variations is thought to link to reward and ‘confirmation bias’ which enhance the sense that the treatment is working.

A similar finding presents a study where pain sensation of subjects was influenced by the cue they received describing the pain versus the actual pain delivered in form of a local heat stimuli. The difference in subjective evaluation guided by the cue and the actual experience is the equivalent of a placebo effect. Subjects of the Met/Met genotype experienced a significantly larger placebo effect in this study compared to subjects of the Val/Val genotype. Val/Met patients fell statistically in the middle of the other two polymorphism groups. (Rongjun Yu et al., (2014) Placebo analgesia and reward processing: Integrating genetics, personality, and intrinsic brain activity. Human Brain Mapping. 35: 4583-4593)

Subjects having one or more COMT polymorphisms may indicate an increased likelihood the subjects would exhibit a placebo effect. In some cases, these subjects may be excluded from the clinical trials or included in clinical trials. Additionally, the presence of one or more COMT polymorphisms in the subjects may suggest that a treatment of the subjects with the COMT polymorphism may be modified. In another embodiment, the treatment may include identifying a subject with a COMT polymorphism, modulating the treatment for the subject based presence or absence of the COMT polymorphism, and administering the modulated treatment. The modulations of the treatment may include adjusting (e.g. increasing or decreasing) a dosage or strength of the treatment administered to the subject depending on the COMT polymorphism. The “dosage,” as used herein, refers to a specified quantity of a treatment and “strength,” as used herein, refers to the concentration of a dosage.

### Clinical Trials

For many classes of medications, such as analgesics, antidepressants, angina treatments, antihistamines and nonsteroidal asthma prophylaxis, well-designed, randomized, placebo-controlled trials (RCTs) often show no difference between drug and placebo. As a consequence, RCTs commonly use various ‘enrichment’ strategies that can selectively exclude participants based on pretreatment response to placebo (placebo run-in) or include them if they respond to drug (predictive enrichment). The enriched subset of patients is then randomized to drug or placebo. For example, in some embodiments, a clinical trial is a placebo-controlled clinical trial where some subjects of the trial receive at least an experimental treatment and other subjects of the trial receive at least a placebo treatment. By depleting placebo responders—or enriching for drug responders—it is expected that the trial will show a larger drug—placebo difference, thus increasing power while decreasing sample size. Many potential threats to the validity of enrichment strategies have been proposed. For example, placebo run-in subjects may experience unblinding side effects once shifted to active drug and, conversely, patients pre-treated with drug then randomized to placebo can experience withdrawal relapse creating a bias against placebo. Probably the most severe criticism is that placebo-run-in methodologies generally fail to adequately and consistently predict patients' predispositions to having a placebo response in the clinical trial.

Given the limitations of current enrichment strategies, coupled with the recent increases in clinical trial costs and placebo-response rates, identifying placebo-response biomarkers to guide enrichment could prove to be a valuable strategy. COMT genetic variants are an efficient and inexpensive way of potentially identifying a significant proportion of placebo responders, thus potentially greatly reducing the clinical time and resources involved in treatment-based enrichment strategies. For example, the exclusion of genetically designated high placebo responders—such as, COMT rs4680 Met/Met—and the inclusion of low placebo responders—such as, Val/Val can greatly reduce the clinical time and resources involved in treatment-based enrichment strategies. Heterozygous genetic variants may represent the largest group; and have an intermediate level of placebo response. Inclusion of COMT genetic variants would be expected to decrease effect sizes of clinical trials. The “effect size,” as used herein, refers to the number of subjects needed in a clinical trial to demonstrate statistical significance of active treatment efficacy over placebo response.

In one aspect, a method of selecting subjects to participate in a clinical trial includes identifying subjects with a catechol-O-methyltransferase (COMT) polymorphism, where the COMT polymorphism modulates a placebo effect in the subjects, and selecting subjects to participate in the clinical trial based on their COMT polymorphism. The COMT polymorphism can be at least one of rs4680, rs4818, rs6269, and rs4633. The COMT polymorphism can also encode a valine/methionine haplotype, a methionine/methionine haplotype, or a valine/valine haplotype. In one embodiment, the COMT polymorphism indicates an increased likelihood the subjects would exhibit a placebo effect. By identifying subjects with the COMT polymorphism, those subject may be excluded or included from a clinical trial or a treatment may be modified depending on the COMT polymorphism.

In another aspect patients with a certain disorder are evaluated in a clinical study for their placebo response and a correlation is established between high placebo responders and their respective COMT polymorphism haplotype. The COMT haplotype, e.g. COMT rs4680 Met/Met or COMT rs4680 Val/Val, that demonstrated the highest correlation to an observed placebo effect is effectively determined to be a predictor for placebo response in the evaluated indication. Patients of this COMT haplotype type can be excluded from future clinical studies for the purpose of reducing the placebo rate in RCTs. The COMT polymorphism can be at least one of rs4680, rs4818, rs6269, and rs4633. The COMT polymorphism can also encode a valine/methionine haplotype, a methionine/methionine haplotype, or a valine/valine haplotype.

In another aspect, after the exclusion of patients with a COMT polymorphism haplotype correlated to high placebo response, the remaining patients are randomized in the various, comparative treatment arms and placebo arms of the clinical trials in ways that result in an equal distribution of the remaining COMT haplotypes in each arm in order to achieve best possible equalization of treatment responders and placebo responders in all arms of the study. The COMT polymorphism can be at least one of rs4680, rs4818, rs6269, and rs4633. The COMT polymorphism can also encode a valine/methionine haplotype, a methionine/methionine haplotype, or a valine/valine haplotype.

In another aspect, patients are randomized in the various, comparative treatment arms and placebo arms of the clinical trials in ways that result in an equal distribution of the COMT haplotypes in each arm in order to achieve best possible equalization of treatment responders and placebo responders in all arms of the study. The COMT polymorphism can be at least one of rs4680, rs4818, rs6269, and rs4633. The COMT polymorphism can also encode a valine/methionine haplotype, a methionine/methionine haplotype, or a valine/valine haplotype.

Treatments

In some embodiments a treatment is administered to a subject. In certain embodiments a treatment is administered to one or more subjects having, suspected of having or at risk of having a disorder, ailment, disease or condition. In certain embodiments a treatment is administered to a subject or group of subjects as part of a clinical trial. In certain embodiments a treatment is administered to a subject or group of subjects selected as a test group or study group. In some embodiments all subjects of a clinical trial, test group or study group are administered a treatment. In some embodiments some subjects, or selected subjects of a clinical trial, test group or study group are administered a treatment. A treatment can comprise administering one or more compounds and/or drugs to one or more subjects. A treatment can comprise administering a therapy to one or more subjects. Non-limiting examples of a therapy include a surgery (minor or major, invasive or non-invasive), physical therapy, administering a physical stimuli (e.g., acupuncture, electrical treatment, sonic treatment, radiation treatment and the like). Any suitable, new, known or experimental compound, drug or therapy can be administered to a sub-population, sub-group, study group or human subject selected by a method described herein. Suitable doses and methods of administering new, known or experimental compounds, drugs and/or therapies to human subjects are often determined (e.g., pre-determined) by scientists and/or medical professionals, which doses and methods are often specific for a particular disorder, ailment, disease or condition.

A treatment is sometimes an experimental treatment. In some embodiments an experimental treatment comprises evaluating a treatment (e.g., drug and/or therapy) that is new or experimental. In certain embodiments the efficacy of a treatment is determined according to the results obtained from testing an experimental treatment. In certain embodiments the efficacy of a treatment is determined according to the results obtained from a clinical trial and/or experimental study. In certain embodiments the effective dosing, effective routes of administration, efficacy, and/or adverse effects (e.g., side effects) of a treatment are determined according to the results of an experimental treatment, clinical trial and/or experimental study.

In certain embodiments evaluating or assessing the results of an experimental treatment comprises determining the presence, absence, description and/or degree of one or more subjective symptoms. The presence, absence, description and/or degree of one or more subjective symptoms can be determined prior to, during or after administration of an experimental treatment. Testing an experimental treatment often comprises evaluating the efficacy of a treatment to reduce or alleviate one or more subjective symptoms.

In certain embodiments an experimental treatment is administered as part of a clinical trial, clinical study or medical study. In some embodiments, administering an experimental treatment comprises administering an experimental drug or therapy. In certain embodiments an experimental treatment is administered to one or more study groups. Some or all subjects of a sub-group or study group, or in certain embodiments some or all sub-groups or study groups may receive an experimental treatment. In some embodiments, an experimental drug or therapy is not administered to all subjects, sub-groups or study groups of a clinical study or medical study.

In some embodiments a placebo treatment is administered. A placebo treatment can be any simulated or otherwise medically ineffectual treatment administered to a subject. A placebo can be any compound or composition that is known or expected to have little or no pharmacological effect on a disorder, disease or condition. In certain embodiments a placebo treatment is administered orally. In certain embodiments a placebo treatment comprises administering a placebo tablet, granule, liquid, capsule, pill, or the like wherein the tablet, granule, liquid, capsule or pill contain an inert ingredient (e.g., ineffective ingredient). A placebo can be administered by any suitable method which is often the same method used to administered an experimental drug or experimental compound that is being evaluated. A placebo can be self-administered or administered by another.

In some embodiments a placebo treatment comprises a mock surgery or mock treatment.

In certain embodiments conducting a clinical or medical trial comprises testing an experimental treatment. In some embodiments a method of testing an experimental treatment comprises administering an experimental treatment to some or all subjects, sub-groups, test groups or study groups and administering a placebo treatment to some or all subjects, sub-groups, test groups or study groups. In certain embodiments a method of testing an experimental treatment comprises administering an experimental treatment to some subjects, sub-groups, test groups or study groups and administering a placebo treatment to other subjects, sub-groups, test groups or study groups where the subjects, sub-groups, test groups or study groups that receive the experimental treatment are different subjects, sub-groups, test groups or study groups than those that received the placebo treatment. In some embodiments a method of testing an experimental treatment comprises excluding some subjects, sub-groups, test groups or study groups from receiving either an experimental treatment or a placebo treatment. In certain embodiments conducting a clinical or medical trial comprises excluding some subjects, sub-groups, test groups or study groups from participating in a clinical or medical trial.

In certain embodiments a study (e.g., a medical study, a clinical trial) comprises testing an experimental drug where a sub-population of subjects is first selected for participation in the study. Subjects of such sub-population are often selected because they have a particular disorder, disease or condition and/or because they are in need of a treatment for a particular disorder, disease or condition. In certain embodiments a sub-population of subjects are selected for participation in a study because they have a particular disorder or disease and the study is designed to test an experimental treatment for the particular disorder or disease. Sometimes subjects of a sub-population of subjects are selected by a random method. In some embodiments, subjects of a sub-population of subjects are selected for participation in a clinical trial according to the presence or absence of one or more COMT polymorphisms.

In certain embodiments, a sub-population of subjects are tested for the presence or absence of one or more COMT polymorphisms. In certain embodiments, the presence or absence of a COMT polymorphism is detected in a group of human subjects (e.g., a sub-population). A sub-population of subjects can be further segregated or divided into two or more sub-groups and/or study groups according to the presence or absence of one or more COMT polymorphisms detected. In some embodiments, subjects of a sub-population that are determined to have one or more COMT polymorphisms are excluded from a study or clinical trial. In some embodiments, subjects of a sub-population that are determined to have one or more COMT polymorphisms are be excluded from one or more study groups or test groups of a study. For example, in certain embodiments a sub-group of subjects determined to have a COMT polymorphism are excluded from one or more, or all study groups of a study (e.g., study groups of a medical or clinical trial). Subjects excluded from a study are often not administered a treatment of any kind (e.g., an experimental treatment or placebo treatment). In certain embodiments, subjects of a subgroup that are determined to have a COMT polymorphisms are distributed among two or more study groups in an attempt to normalize or minimize placebo effects on a medical or clinical study. In certain embodiments, subjects of a subgroup that are determined to have a COMT polymorphisms are distributed equally or about equally among at least two study groups in an attempt to normalize or minimize placebo effects on a medical or clinical study. In some embodiments, subjects of a sub-group that have a COMT polymorphism and subjects that do not have a COMT polymorphism are distributed equally or about equally among at least two study groups in an attempt to normalize or minimize placebo effects. In some embodiments, distributing subjects about equally among one or more groups, sub-groups or study groups includes a random distribution of the subjects, where the distribution is not always an equal distribution. In some embodiments, distributing subjects about equally among groups comprises a randomization method and any suitable randomization method can be used.

In certain embodiments, subjects that are homozygous and/or heterozygous for a COMT polymorphism are excluded from a study. For example, in some embodiments subjects that are homozygous for an A allele of an rs4680 polymorphism are excluded from a study. In some embodiments subjects that are heterozygous for an A allele of an rs4680 polymorphism are excluded from a study. In some embodiments subjects that are heterozygous and subjects that are homozygous for an A allele of an rs4680 polymorphism are excluded from a study.

In certain embodiments, subjects that are homozygous and/or heterozygous for a COMT polymorphism are distributed among study groups of a study (e.g., study groups that receive a placebo treatment, study groups that receive an experimental treatment, control groups and the like). For example, in some embodiments subjects that are homozygous for an A allele of an rs4680 polymorphism are identified and distributed among the study groups that are used for a study. In some embodiments subjects that are heterozygous for an A allele of an rs4680 polymorphism are identified and distributed among the study groups that are used for a study. In some embodiments subjects that are heterozygous and subjects that are homozygous for an A allele of an rs4680 polymorphism are identified and distributed among the study groups that are used for a study. Subjects of a sub-group can be distributed by any suitable method among study groups of a study. For example, subjects of a sub-group can be distributed randomly, evenly and/or equally among study groups of a study.

Each study group or test group of a study is often designated to receive one or more distinct treatments. For example some study groups may be administered an experimental treatment, some may receive a placebo treatment and some may not receive a treatment. The design of certain clinical and/or medical studies can be quite complex involving 2 or more, 3 or more, 5 or more, 10 or more or 20 or more study groups.

In the case of irritable bowel syndrome, diabetes, autoimmune disorders, inflammation, neurological disorders, acute pain, chronic pain, cancer, cancer treatments, allergies, depression, migraines, addiction, obesity, cardiovascular disorders and other disorders, syndromes, or diseases, the placebo responders identified by their COMT genotype could receive less of the active pharmacological treatment thus limiting their negative side-effects while still maintaining their placebo response. Alternatively patients identified as non-placebo responders may be candidates for a stronger more focused pharmacological intervention. In the case of clinical conditions with more objective outcomes, such as Parkinson's, asthma, addiction, arthritis, angina and diabetes treatment of placebo responders with drugs that interact with dopamine or other catecholamine pathways could be modified based on the interaction between the treatment and the patient's COMT polymorphism in order to mitigate potential negative interactions or promote positive interactions between the treatment and the catecholamine mediated pathway.

In another aspect, methods for treating a subject are disclosed. One embodiment can include determining a catechol-O-methyltransferase (COMT) polymorphism in the subject, identifying a treatment for the subject based on the COMT polymorphism, and administering the treatment to the subject. The method can also include obtaining a nucleic acid sample from the subject and analyzing the nucleic acid sample for the COMT polymorphism. The method can include obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing the sample for the COMT polymorphism.

Also disclosed is a method for determining a treatment dosage by determining a catechol-O-methyltransferase (COMT) polymorphism in a subject and modulating the treatment dosage administered to the subject based on the COMT polymorphism. The subject can be in a clinical trial and the dosage can be modulated depending on the COMT polymorphism. The method can also include obtaining a nucleic acid sample from the subject and analyzing the nucleic acid sample for the COMT polymorphism. The method can include obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing the sample for the COMT polymorphism.

Identification of the treatment can include modulating the treatment for the subject with the COMT polymorphism and administering the modulated treatment and/or identifying an alternative treatment for the subject with the COMT polymorphism, and administering the treatment to the subject. Additionally, the treatment can be adjusted by administering a modulated, e.g. increased, decreased, or in combination with another treatment, dosage of the treatment to the subject depending on the COMT polymorphism.

Diseases

Studies investigating brain activity associated with placebo response in pain point to catecholamines such as dopamine as a possible integrator of the placebo response. Dopamine and other catecholamine are packaged into presynaptic vesicles and released into the synaptic cleft upon depolarization. Dopamine is cleared from the synapse either by the dopamine reuptake transporter (DAT), or degradation by monoamine oxidases A and B, or catechol-O-methyltransferase (COMT). Whereas reuptake is the primary mechanism of dopamine clearance in the striatum, in the prefrontal cortex, where monoamine oxidase and DAT is less abundant, COMT activity is critical in regulating prefrontal dopamine and other catecholamine signaling.

### Irritable Bowel Syndrome (IBS)

Irritable bowel syndrome (IBS) is a common gastrointestinal disorder affecting 10 to 15% of North Americans is characterized by abdominal pain or discomfort associated with altered bowel function, bloating, and a sensation of incomplete evacuation after bowel movements. IBS is a condition known to have a high placebo response rate and meta-analyses report an average placebo induced global improvement of approximately 40%.

The COMT allele, such as the Met/Met genotype, can be a potential marker for placebo responders in disease. The number of COMT major or minor alleles may correspond to COMT activity. This may, in turn, correspond to dopamine or other catecholamine availability in the prefrontal cortex that would relate to placebo responses. Further, a relationship may exist between number of a given type of COMT allele and placebo response. Furthermore the finding that particular genotypes are associated with a particular outcome, positive or negative, in groups administered a placebo treatment can be a predictor of a placebo effect.

In one aspect, a subject with irritable bowel syndrome, being of the COMT haplotype Met/Met may receive traditional pharmacological treatment with augmented placebo treatment facilitated by the therapeutic encounter with or without non-traditional, non-pharmacological interventions such as acupuncture, massage, placebo pills or similar.

Another aspect can include the tapering off of traditional pharmacological treatments during or after a placebo or non-pharmacological treatment administration in patients with the COMT polymorphism haplotype that responds favorably to placebo treatment.

In one aspect, treating a subject has, is suspected of having, or is at risk for developing irritable bowel syndrome is disclosed. The method can include determining a catechol-O-methyltransferase (COMT) polymorphism in the subject, identifying a treatment for the subject based on the COMT polymorphism, and administering the treatment to the subject.

Another aspect can include determining a treatment dosage for a subject with, suspected of having, or at risk for developing irritable bowel syndrome. The catechol-O-methyltransferase (COMT) polymorphism in a subject can be determined, and the treatment dosage administered to the subject can be modulated based on the COMT polymorphism. The modulation can include increasing or decreasing the dosage for the subject with the COMT polymorphism, identifying an alternative treatment for subject with the COMT polymorphism.

Functional diseases such as IBS, chronic fatigue and Fibromyalgia; diseases related to the pathophysiology of dopamine such as depression and schizophrenia; and disease related to the pathophysiology of other catecholamines including cardiovascular and metabolic diseases such as coronary artery disease and diabetes; may be affected by the COMT mediated placebo response. Screening patients for their COMT polymorphism and selecting the group that responds most favorably to placebo enables the caregiver to augment the pharmacological treatment effect with placebo treatments or non-pharmacological treatments that would elicit an additive placebo effect in these patients. Drug dosages and treatment strengths may be tapered down in these patients.

### Acute or Chronic Pain

Acute or chronic pain as results of injury, surgery or disease is experienced at some point in life by every person. Effective analgesics are available, however they have limitations due to tolerability issues, side effects and dependency issues. For this reason many patients, especially those with chronic pain are underserved. The presented data suggest that pain sensation is highly susceptible to placebo with subjects of the Met-Met haplotype responding significantly more to placebo stimuli. Successful augmentation of pain treatment with placebo, including adjustment of the therapeutic encounter, or non-pharmacological treatments is therefore a feasible alternative to increasing doses or increasing strength of pain medication. Such placebo augmented treatment would spare the patients the side effects of higher doses or stronger pain medication and may even allow tapering of the medication.

### Other Disorders, Syndromes or Diseases

In one aspect, treating a subject with, suspected of having, or at risk for developing at least one of autoimmune disorders, inflammation, neurological disorders, chronic pain, cancer, cancer treatments, allergies, depression, migraines, schizophrenia, addiction, obesity, and any combination of other disorders, syndromes, and diseases is disclosed. Another aspect can include determining a treatment dosage for a subject with, suspected of having, or at risk for at least one of autoimmune disorders, inflammation, neurological disorders, schizophrenia, chronic pain, cancer, cancer treatments, allergies, depression, migraines, addiction, obesity, and any combination of other disorders, syndromes, and diseases.

### Detection Methods, Assays and Kits

In one aspect, methods and materials for a diagnostic kit and assay are included to determine the COMT polymorphism. The kit or assay can include materials for obtaining a nucleic acid sample from the subject and reagents for analyzing the nucleic acid sample for the COMT polymorphism. The kit can also include materials for obtaining a sample, such as a blood, urine, ascites, cerebrospinal fluid, bronchial lavage, oral washings and sputum, Pap smears, tissue biopsies or organs, bile, fecal matter, or other bodily fluids, tissues or parts from the subject and analyzing these samples for the COMT polymorphism.

The presence or absence of a COMT polymorphism can be detected by a suitable method, assay or by a method described herein. In some embodiments a COMT polymorphism is detected at the genetic level (e.g., detection of a COMT polymorphism in genomic DNA or mRNA) using a suitable technique. In some embodiments a COMT polymorphism is detected at the protein level (e.g., detection of a COMT polymorphism in COMT protein) using a suitable technique. For example, in some aspects, a protein or peptide assay for determining a COMT polymorphism in a subject can be used. In certain embodiments, detecting the presence or absence of a COMT polymorphism comprises detecting an amino acid mutation (e.g., an amino acid substitution, deletion or insertion) in a COMT polypeptide. In certain embodiments, detecting the presence of a COMT polymorphism comprises detecting the presence of methionine 158 in a COMT polypeptide and/or detecting the absence of valine 158 in a COMT polypeptide. In certain embodiments, detecting the absence of a COMT polymorphism comprises detecting the absence of methionine 158 in a COMT polypeptide and/or detecting the presence of valine 158 in a COMT polypeptide. In certain embodiments, a COMT polymorphism is detected with the use of at least one antibody specific for a COMT polymorphism. In certain embodiments a kit for detecting a COMT polymorphism comprises at least one antibody specific for detecting a COMT polymorphism. The assay can also include reagents for measuring or comparing catechol-O-methyltransferase enzymatic activity. The assay can further include reagents for analyzing catechol-O-methyltransferase protein. Non-limiting example of methods that can be used to detect a COMT polymorphism include immunohistochemistry, a suitable immune assay (e.g., an enzyme-linked immunosorbent assay (ELISA)), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, the like or a combination thereof. Thus a kit or assay for detection of a COMT polymorphism can include reagents specific for any of immunohistochemistry, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, or a combination thereof. In some embodiments a COMT polymorphism is detected by analyzing the expression, modifications and/or conformational changes of a COMT protein or variant thereof (e.g., a variant resulting from a polymorph). For example, in certain embodiments an assay can be configured to detect phosphorylation, glycosylation, carboxylation, methylation, lipid modification, ubiquitination, myristoylation, conformation, conformational changes in protein folding, monomerization and/or polymerization, or other conformational states of a COMT protein. In some embodiments a kit or assay can comprise reagents for detecting modifications and/or conformational changes of COMT protein or other biomarkers, such as phosphorylation, glycosylation, methylation, lipid modification, ubiquitination, myristoylation state, conformational change in protein folding, monomerization and/or polymerization, and other conformational states of the protein.

A suitable sample can be obtained from a subject for analysis. The sample can include, for example, nucleic acids, proteins, peptides, precursors, lipids, carbohydrates, metabolites, and other COMT polymorphism biomarkers to be separated/isolated/purified from bodily fluids, cells, or tissues of a subject. Any suitable method can be used to isolate, separate, and/or purify a COMT protein, mRNA or DNA. Cell separation/isolation/purification methods can be used to isolate a diagnostic sample for use with a diagnostic kit. A skilled artisan can use any known cell separation/isolation/purification techniques to isolate the diagnostic sample from the subject's sample. Exemplar techniques include, but are not limited to, using antibodies, flow cytometry, fluorescence activated cell sorting, filtration, gradient-based centrifugation, elution, microfluidics, magnetic separation technique, fluorescent-magnetic separation technique, nanostructure, quantum dots, high throughput microscope-based platform, or a combination thereof.

In certain aspects described herein, analytes include nucleic acids, proteins, lipids, carbohydrates, metabolites, or any combinations of these. In certain aspects of the methods described herein, biomarkers include nucleic acids, proteins, precursors, lipids, carbohydrates, metabolites, or any combinations of these. As used herein, the term “nucleic acid” is intended to include DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNAs), microRNA (miRNAs), small nucleolar RNA (snRNAs), ribosomal RNA (rRNA), transfer RNA (tRNA), small interfering RNA (siRNA), heterogeneous nuclear RNAs (hnRNA), or small hairpin RNA (shRNA).

A kit (e.g., a diagnostic kit) can be used to detect the presence, absence, or a difference in genetic alleles. In some embodiments a kit can include suitable nucleic acids or suitable reagents (e.g., at least one primer or probe specific for a COMT polymorphism and hybridization reagents, e.g., one or more primers configured to amplify a portion of a gene or cDNA suspected of having a COMT polymorphism) to determine the presence or absence of a COMT polymorphism in a subject. In certain embodiments, a difference between different profiles detected can refer to different gene copy numbers, genetic alleles, different DNA, RNA, proteins, lipids, or carbohydrate expression levels, different DNA methylation states, different DNA acetylation states, and different protein modification states that result from different genetic profiles.

As used herein, a “profile” of a marker, e.g. COMT, of a disease or condition can broadly refer to any information concerning the marker. This information can be either qualitative (e.g., presence or absence) or quantitative (e.g., levels, copy numbers, or dosages). In some embodiments, a profile of a marker can indicate the absence of this marker. The profile can be a nucleic acid (e.g., DNA or RNA) profile, a protein profile, a lipid profile, a carbohydrate profile, a metabolite profile, or a combination thereof. A “marker” as used herein generally refers to an analyte which is differentially detectable and is indicative of the presence of a disease or condition. An analyte is differentially detectable if it can be distinguished quantitatively or qualitatively. Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

A nucleic acid profile can be, without limitation, a genotype, a genotypic profile, a single nucleotide polymorphism profile, a gene mutation profile, a gene copy number profile, a DNA methylation profile, a DNA acetylation profile, a chromosome dosage profile, a gene expression profile, or a combination thereof.

A COMT polymorphism and/or a nucleic acid profile can be determined by a suitable method known in the art to detect genotypes, single nucleotide polymorphisms, gene mutations, gene copy numbers, DNA methylation states, DNA acetylation states, chromosome dosages and the like. In certain embodiments, detecting the presence or absence of a COMT polymorphism comprises detecting a polymorphism in one or more alleles of a COMT gene. In certain embodiments, detecting the presence of a COMT polymorphism comprises detecting the presence or absence of one or more polymorphisms of Table 1 in a COMT gene. In certain embodiments, detecting the presence of a COMT polymorphism comprises detecting the presence or absence of rs4680, rs4818, rs6269, and/or rs4633 in a COMT gene. Non-limiting examples of methods that can be used to detect the presence or absence of a COMT polymorphism, COMT haplotype and/or COMT genotype profile include a polymerase chain reaction (PCR) analysis, sequencing analysis (e.g., next generation sequencing), electrophoretic analysis, restriction fragment length polymorphism (RFLP) analysis, Northern blot analysis, quantitative PCR, reverse-transcriptase-PCR analysis (RT-PCR), allele-specific oligonucleotide hybridization analysis, comparative genomic hybridization, heteroduplex mobility assay (HMA), single strand conformational polymorphism (SSCP), denaturing gradient gel electrophoresis (DGGE), RNAase mismatch analysis, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), surface plasmon resonance, Southern blot analysis, in situ hybridization, fluorescence in situ hybridization (FISH), chromogenic in situ hybridization (CISH), immunohistochemistry (IHC), microarray, comparative genomic hybridization, karyotyping, multiplex ligation-dependent probe amplification (MLPA), Quantitative Multiplex PCR of Short Fluorescent Fragments (QMPSF), microscopy, methylation specific PCR (MSP) assay, Hpall tiny fragment Enrichment by Ligation-mediated PCR (HELP) assay, radioactive acetate labeling assays, colorimetric DNA acetylation assay, chromatin immunoprecipitation combined with microarray (ChIP-on-chip) assay, restriction landmark genomic scanning, Methylated DNA immunoprecipitation (MeDIP), molecular break light assay for DNA adenine methyltransferase activity, chromatographic separation, methylation-sensitive restriction enzyme analysis, bisulfate-driven conversion of non-methylated cytosine to uracil, methyl-binding PCR analysis, the like or combinations thereof.

As used herein, the term “sequencing” is used in a broad sense and refers to any technique known in the art that allows the order of at least some consecutive nucleotides in at least part of a nucleic acid to be identified, including without limitation at least part of an extension product or a vector insert. Non-limiting examples of sequencing techniques include direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and a combination thereof. In some embodiments, sequencing comprises an detecting the sequencing product using an instrument, for example but not limited to an ABI PRISM® 377 DNA Sequencer, an ABI PRISM® 310, 3100, 3100-Avant, 3730, or 373Oxl Genetic Analyzer, an ABI PRISM® 3700 DNA Analyzer, or an Applied Biosystems SOLiD® System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

In certain embodiments, methods of this invention further comprise comparing the identified difference of the disease or condition-specific markers to a repository of at least one markers known in the art. Such comparison can further confirm the presence of the disease or condition. In some embodiments, the repository of the known markers can be obtained by data mining. The term “data mining”, as used herein, refers to a process of finding new data patterns, relations, or correlations derived from the known data of the databases and of extracting practicable information in the future. Typically a computer-based system can be trained on data to perform the data mining, e.g., to classify the input data and then subsequently used with new input data to make decisions based on the training data. These systems include, but are not limited, expert systems, fuzzy logic, non-linear regression analysis, multivariate analysis, decision tree classifiers, and Bayesian belief networks.

In further embodiments of the invention, a protein profile can be a protein expression profile, a protein activation profile, or a combination thereof. In some embodiments, a protein activation profile can comprise determining a phosphorylation state, a glycosylation state, methylation state, lipid modification, an ubiquitination state, a myristoylation state, conformational change in protein folding, monomerization and/or polymerization, and a conformational state of the protein.

A protein profile can be detected by any methods known in the art for detecting protein expression levels, protein phosphorylation state, protein ubiquitination state, protein myristoylation state, or protein conformational state. In some embodiments, a protein profile can be determined by an immunohistochemistry assay, an enzyme-linked immunosorbent assay (ELISA), in situ hybridization, chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APP I-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassay, microscopy, microfluidic chip-based assays, surface plasmon resonance, sequencing, Western blotting assay, or a combination thereof.

### Pharmaceutical Applications

As used herein, “administering” or “administration of” a compound or an agent to a subject with a particular COMT genetic profile can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasal (by inhalation), intraspinally, intracerebrally, rectally, vaginally, and transdermally (by absorbtion, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. In some embodiments, a compound or an agent is administered orally, e.g., to a subject by ingestion, or intravenously, e.g., to a subject by injection. In some embodiments, the orally administered compound or agent is in an extended release or slow release formulation, or administered using a device for such slow or extended release.

All publications, patents and patent applications cited herein, whether supra or infra, are hereby incorporated by reference in their entirety. As used in this specification and the appended claims, the singular forms “a,” “an,” and “the” include plural references unless the content clearly dictates otherwise. The terms used in this invention adhere to standard definitions generally accepted by those having ordinary skill in the art. In case any further explanation might be needed, some terms have been further elucidated below.

### Representative Sequences

Shown below is a representative nucleic acid sequence of mRNA transcript variant X1 of Homo sapiens catechol-O-methyltransferase (COMT)(NCBI Reference Sequence: XM_{—}005261229.1). This sequence was derived from genomic sequence NT 011520.13 annotated using a gene prediction method. The coding sequence is shown from nucleotide 502 to nucleotide 1317.

The location of the rs4680 polymorphism is at nucleotide number 973 where the G allele is shown. The location of the rs4633 polymorphism is at nucleotide number 687 where the C allele is shown. The location of the rs6269 polymorphism is at nucleotide 404 where the A allele is shown. The location of the rs4818 polymorphism is at nucleotide 909 where the C allele is shown.

The location of additional polymorphisms listed in Table 1 are described in NCBI Reference Sequence: XM_{—}005261229.1.

COMT mRNA Transcript Variant X1

### NCBI Reference Sequence: XM_{—}005261229.1

### LOCUS: XM_{—}005261229

| | |
|---|---|
| 1 | gcgccaccgc cattgccgcc atcgtcgtgg ggcttctggg |
| | gcagctaggg ctgcccgccg |
| 61 | cgctgcctgc gccggaccgg ggcgggtcca gtcccgggcg |
| | ggccgtcgcg ggagagaaat |
| 121 | aacatctgct ttgctgccga gctcagagga gaccccagac |
| | ccctcccgca gccagagggc |
| 181 | tggagcctgc tcagaggtgc tttgaagagg atccctgggc |
| | tgcctgggga agcacccagg |
| 241 | gccagggagt gtgaccctgc aggctccaca caggactgcc |
| | agaggcacac acctgctctg |
| 301 | tctacccgag ggcaccagag ggcacgagaa ggctggctcc |
| | ctggcgctga cacgtcaggc |
| 361 | aactgaggca caaggctggc atttctgaac cttgcccctc |
| | tgcaaacaca agggggcgat |
| 421 | ggtggcactc caagcaaagg ggcgtgtggg tgctgcagga |
| | ggagcacaga gcactggcgc |
| 481 | ccctcccctc ccgccctgca gatgccggag gccccgcctc |
| | tgctgttggc agctgtgttg |
| 541 | ctgggcctgg tgctgctggt ggtgctgctg ctgcttctga |
| | ggcactgggg ctggggcctg |
| 601 | tgccttatcg gctggaacga gttcatcctg cagcccatcc |
| | acaacctgct catgggtgac |
| 661 | accaaggagc agcgcatcct gaaccacgtg ctgcagcatg |
| | cggagcccgg gaacgcacag |
| 721 | agcgtgctgg aggccattga cacctactgc gagcagaagg |
| | agtgggccat gaacgtgggc |
| 781 | gacaagaaag gcaagatcgt ggacgccgtg attcaggagc |
| | accagccctc cgtgctgctg |
| 841 | gagctggggg cctactgtgg ctactcagct gtgcgcatgg |
| | cccgcctgct gtcaccaggg |
| 901 | gcgaggctca tcaccatcga gatcaacccc gactgtgccg |
| | ccatcaccca gcggatggtg |
| 961 | gatttcgctg gcgtgaagga caaggtcacc cttgtggttg |
| | gagcgtccca ggacatcatc |
| 1021 | ccccagctga agaagaagta tgatgtggac acactggaca |
| | tggtcttcct cgaccactgg |
| 1081 | aaggaccggt acctgccgga cacgcttctc ttggaggaat |
| | gtggcctgct gcggaagggg |
| 1141 | acagtgctac tggctgacaa cgtgatctgc ccaggtgcgc |
| | cagacttcct agcacacgtg |
| 1201 | cgcgggagca gctgctttga gtgcacacac taccaatcgt |
| | tcctggaata cagggaggtg |
| 1261 | gtggacggcc tggagaaggc catctacaag ggcccaggca |
| | gcgaagcagg gccctgactg |
| 1321 | cccccccggc ccccctctcg ggctctctca cccagcctgg |
| | tactgaaggt gccagacgtg |
| 1381 | ctcctgctga ccttctgcgg ctccgggctg tgtcctaaat |
| | gcaaagcaca cctcggccga |
| 1441 | ggcctgcgcc ctgacatgct aacctctctg aactgcaaca |
| | ctggattgtt cttttttaag |
| 1501 | actcaatcat gacttcttta ctaacactgg ctagctatat |
| | tatcttatat actaatatca |
| 1561 | tgttttaaaa atataaaata gaaattaaga atctaaatat |
| | ttagatataa |

Shown below is a representative polypeptide sequence of Homo sapiens catechol-O-methyltransferase (COMT) as translated from NCBI Reference Sequence XM_{—}005261229.1. The amino acid sequence is similar to UniProt P21964. Amino acids 1 to 271 are shown.

The location of the rs4680 polymorphism is at amino acid number 158 where the Val158 allele is shown. The location of the rs6267 polymorphism is at amino acid number 72 where the Alanine allele is shown. The presence of the rs6267 polymorphism results in a substitution of Alanine at position 72 with Serine at position 72. The Serine 72 variant is correlated with reduced COMT enzyme activity. Amino acids 1-50 are missing in the soluble form of this protein. The position of additional polymorphisms (amino acid substitutions) can be found online at UniProt (URL: http://www.uniprot.org/uniprot/P21964, accessed Oct. 13, 2014). polymorphisms listed in Table 1 are described in NCBI Reference Sequence: XM_{—}005261229.1.

### COMT Polypeptide

MPEAPPLLLA AVLLGLVLLV VLLLLLRHWG WGLCLIGWNE
FILQPIHNLL MGDTKEQRIL NHVLQHAEPG NAQSVLEAID
TYCEQKEWAM NVGDKKGKIV DAVIQEHQPS VLLELGAYCG
YSAVRMARLL SPGARLITIE INPDCAAITQ RMVDFAGVKD
KVTLVVGASQ DIIPQLKKKY DVDTLDMVFL DHWKDRYLPD
TLLLEECGLL RKGTVLLADN VICPGAPDFL AHVRGSSCFE
CTHYQSFLEY REVVDGLEKA IYKGPGSEAG P*

### EXAMPLES

### Example 1

### Irritable Bowel Syndrome

### Materials and Methods

A randomized clinical trial investigating placebo effects in IBS patients (Trial registration—NCT00065403) was conducted. Details of the design and outcomes of the trial are provided elsewhere. The 3-week trial enrolled 262 patients (75% women) 8 years and diagnosed by IBS Rome II criteria score of >150 on the Irritable Bowel Syndrome Symptom Severity Scale (IBS-SSS).

Patients were randomized to one of three treatment arms: (1) no-treatment control (“waitlist); (2) placebo acupuncture (“limited”); (3) placebo acupuncture plus a supportive patient-provider (“augmented”). A validated sham acupuncture device was used to deliver placebo acupuncture in 20 minute sessions, twice weekly for three weeks.

A subgroup of patients (n=112) gave consent for genetic analysis from blood samples included in this study. The Institutional Review Board at Beth Israel Deaconess Medical Center (Boston, Mass.) approved the main study and the genetic follow-up study presented here. All studies were conducted in accordance with the Declaration of Helsinki. Participants provided written consent for this genetic study. The ethics committee approved this procedure.

Three validated IBS research measures from the previous trial, which assessed clinical outcomes, were used in this study. The primary outcome measure was the IBS-SSS, which consists of five 100-point scales (abdominal pain severity, abdominal pain frequency, abdominal distention severity, dissatisfaction with bowel habits, and quality of life disruption) that contribute equally to the final score, yielding a theoretical range of 0-500. Higher scores reflected a more severe condition. IBS-SSS was measured at baseline and after 3 weeks of treatment. Change in IBS-SSS was determined by subtracting 3-week IBS-SSS score from baseline IBS-SSS. We selected IBS-SSS as our primary outcome because the two secondary measures, Adequate Relief (AR) and the Global Improvement Scale (GIS) did not have baseline measures. Adequate Relief was assessed by a single dichotomous question at 3-weeks, which asked: “Over the past week have you had adequate relief of your IBS symptoms?”. The GIS asked: “Compared to the way you felt before you entered the study, have your IBS symptoms over the past 7 days been: (1)=substantially worse, (2)=moderately worse, (3)=slightly worse, (4)=no change, (5)=slightly improved, (6)=moderately improved, or (7)=substantially improved”. These latter two measures were considered secondary because they do not have baseline assessments, thus opening the door to regression artifacts. To mitigate this problem, we controlled for IBS-SSS baseline scores in analyses of AR and GIS.

Of the 262 original study participants, 112 gave consent for genetic screening. Eight patients missing IBS-SSS data at 3-weeks were excluded from the analyses. Two additional patients were missing data for AR and GIS and were excluded from analysis of AR and GIS. Genomic DNA was extracted from whole blood using Qiagen Blood kit (Valencia, Calif.) following the manufacturer's protocol. Based on the association of COMT SNP rs4633 with COMT expression and activity, this SNP was genotyped in addition to rs4680 (Val158met). TaqMan SNP Genotyping assays for rs4680 (Val158met) and rs4633 were purchased from Applied Biosystems, (Foster City, Calif.). Quantitative PCR was performed at the Biopolymers Facility at Harvard Medical School, (Boston, Mass.) following the manufacturer's protocol on an Applied Biosystems 7900HT instrument, using SDS version 2.4 software.

Hardy-Weinberg Equilibrium (HWE) and Linkage Disequilibrium were calculated using the Online Encyclopedia for Genetic Epidemiology studies. Statistical analyses were performed using IBM SPSS Statistics version 20 (Chicago, Ill.).

An additive genetic model was used to investigate the linear effect of increases in the presence of the COMT met allele. A variable was created, “COMT genotype”, that coded each patient's Val158met genotype as follows: 1=Met/Met; 0=Val/Met; −1=Val/Val. Using standard coding for polynomial trends, multiple regression was used to examine linear and quadratic effects of COMT genotype (number of met alleles) on placebo responses as measured by changes from baseline IBS-SSS, and on AR and GIS. Initial disease severity was controlled for by including baseline IBS-SSS as a covariate in the regression models. In addition, variables were created to test for linear and quadratic effects of treatment, conceiving these as ascending “doses” of non-specific effects (waitlist, limited placebo, augmented placebo) to test for interactions between COMT genotype effects and treatment received.

The clinical and demographic characteristics of the subset of genotyped patients (n=104) relative to the original clinical trial (n=262) are shown in Table 2. Age, gender, race and marital status of the genotyped patients did not differ significantly from the distribution in the original study; duration, IBS type and baseline IBS-SSS were also similar. Eighty percent of the genotyped patients were women and 94% were white. Furthermore there were no significant differences in demographics and disease characteristics of genotyped patients across the COMT Val158met genotypes. The number of patients genotyped and analyzed from each treatment arm (waitlist, 29%; limited, 32%; and augmented, 39%) was similar to the overall distribution in the original trial (waitlist, 33%; limited, 34%; and augmented, 33%).

TABLE 2
Baseline characteristics of genotyped IBS patients compared
to participants in the parent randomized clinical trial

| | All IBS | Genotyped | | | | | |
|---|---|---|---|---|---|---|---|
| | patients* | patients | p | COMT rs4680 Genotype | | | p |
| Characteristics | (n = 262) | (n = 104) | value | Met/Met | Met/Val | Val/Val | value |

Demographics

| | | | | | | | |
|---|---|---|---|---|---|---|---|
| Age in years (SD) | 38 (14) | 37 (13) | 0.53 | 40 (12) | 36 (13) | 38 (14) | 0.44 |
| Women (%) | 218 (75) | 83 (80) | 0.51 | 16 (80) | 45 (81) | 22 (76) | 0.82 |
| White (%) | 252 (88) | 98 (94) | 0.17 | 20 (100) | 52 (95) | 26 (89) | 0.20 |
| Married/living together (%) | 101 (45) | 32 (39) | 0.42 | 8 (42) | 16 (41) | 8 (33) | 0.79 |

IBS symptoms and baseline characteristics

| | | | | | | | |
|---|---|---|---|---|---|---|---|
| Type of IBS | | | 0.56 | | | | 0.80 |
| Constipation (%) | 74 (27) | 35 (34) | | 5 (25) | 21 (39) | 9 (31) | |
| Diarrhea (%) | 65 (23) | 18 (17) | | 4 (20) | 8 (15) | 6 (21) | |
| Mixed (%) | 139 (50) | 40 (49) | | 11 (55) | 25 (46) | 14 (48) | |
| Disease for >5 years (%) | 103 (35) | 63 (61) | 0.18 | 12 (60) | 34 (62) | 17 (59) | 0.96 |
| Baseline IBS-SSS (SD) | 273 (73) | 270 (66) | 0.72 | 291 (78) | 261 (64) | 272 (61) | 0.23 |

Based on its association with rs4680, COMT expression and enzymatic activity rs4633 was also genotyped. Rs4633 was found to be in strong linkage disequilibrium with rs4680 (D′=0.94 and r2=0.9), such that the two SNPs were almost perfectly correlated: Met/Met, Val/Met and Val/Val of rs4680 corresponded to the T/T, T/C and C/C of rs4633. Therefore we focused on COMT Val158met in further analyses. The minor allele frequency of COMT Val158met was 0.46 and the SNP was in Hardy-Weinberg Equilibrium (p=0.502). See Table 3.

TABLE 3
Characterization of Selected Polymorphisms

| SNP | Alleles | Minor Allele | MAF | Mutation Type | HWE p |
|---|---|---|---|---|---|
| rs4680 | A/G | A | 0.46 | Mis-sense Mutation | >.05 |
| | | | | Transition Substitution | |
| rs4633 | C/T | T | 0.47 | Silent Mutation | >.05 |
| | | | | Transition Substitution | |
| rs4818 | G/C | G | 0.44 | Transversion Substitution | >.05 |

In this study, IBS Symptom Severity Scale (IBS-SSS) was a priori primary clinical outcome. IBS-SSS is a multidimensional measure that captures the full spectrum of IBS disease including abdominal pain severity, abdominal pain frequency, abdominal distention severity, dissatisfaction with bowel habits, and disruption in quality of life and has a theoretical range of 0-500.

Both linear and quadratic effects of COMT alleles and treatment arm (waitlist, limited, augmented) were tested. As there were no significant main effects or interactions involving quadratic tests, results reported here are for a trimmed model with only linear effects and interactions included.

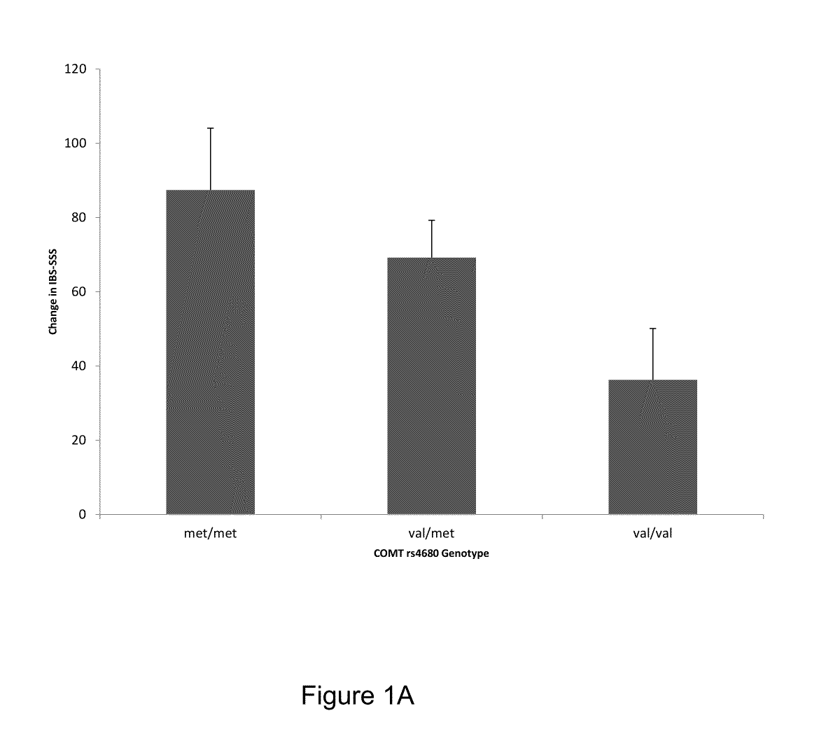

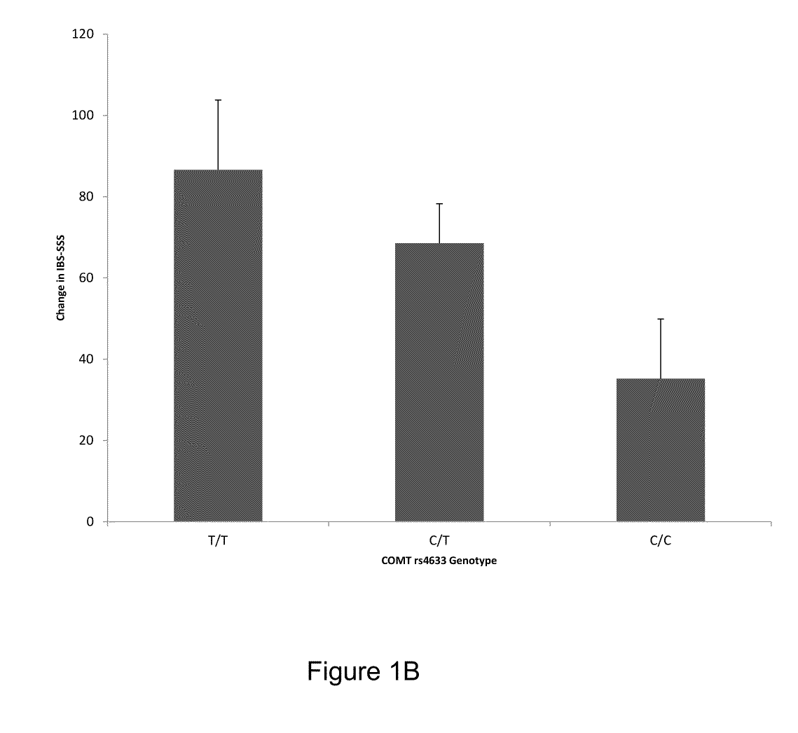

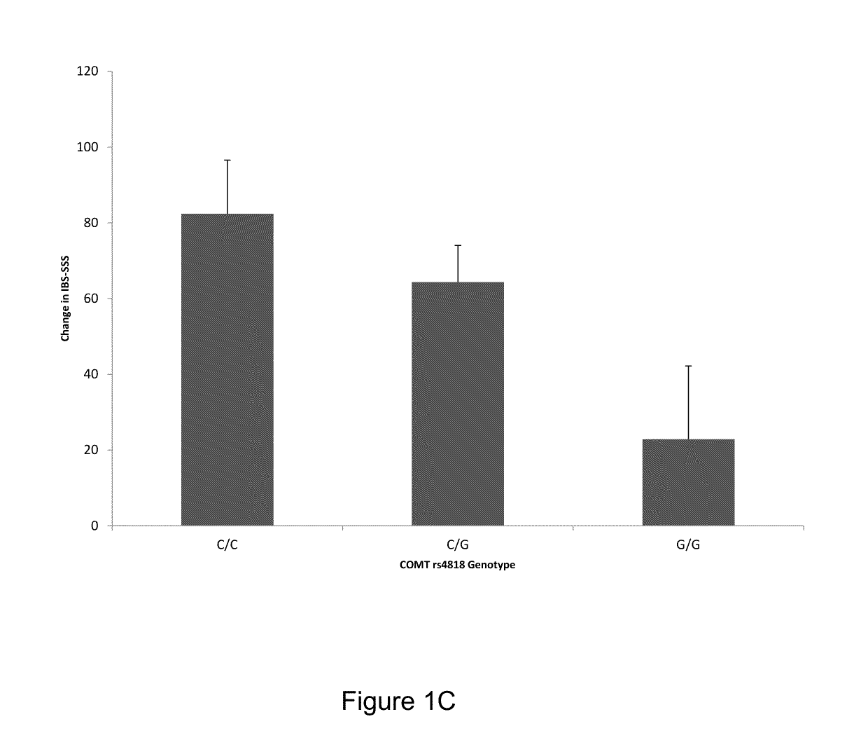

Change in IBS-SSS was associated with a main effect of COMT genotype (number of met alleles) (FIGS. 1A-1C). Number of COMT Val158met met alleles showed a significant linear effect on IBS-SSS (beta=0.17; p=0.032). Patients with the Met/Met genotype had the greatest level of improvement whereas Val/Val patients had the least, and Val/Met patients were intermediate (FIG. 1A). As in the original study, there was a main effect of treatment arm on improvement, with patients in the augmented treatment improving most and those on waitlist improving least (beta=0.19, p=0.019).

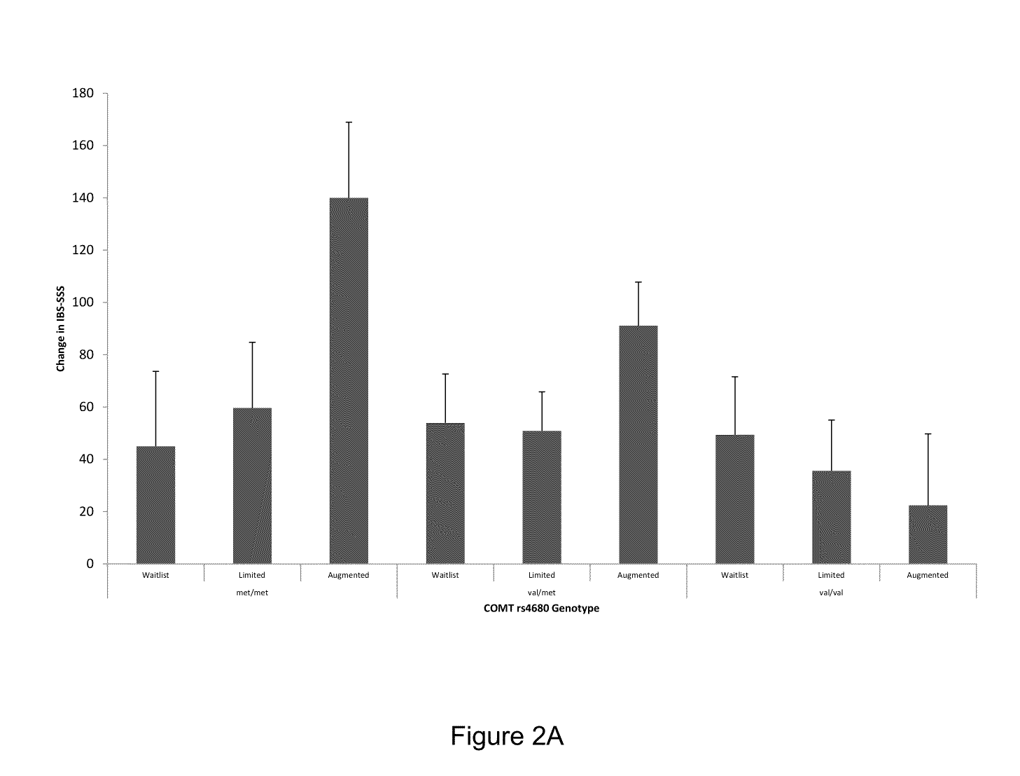

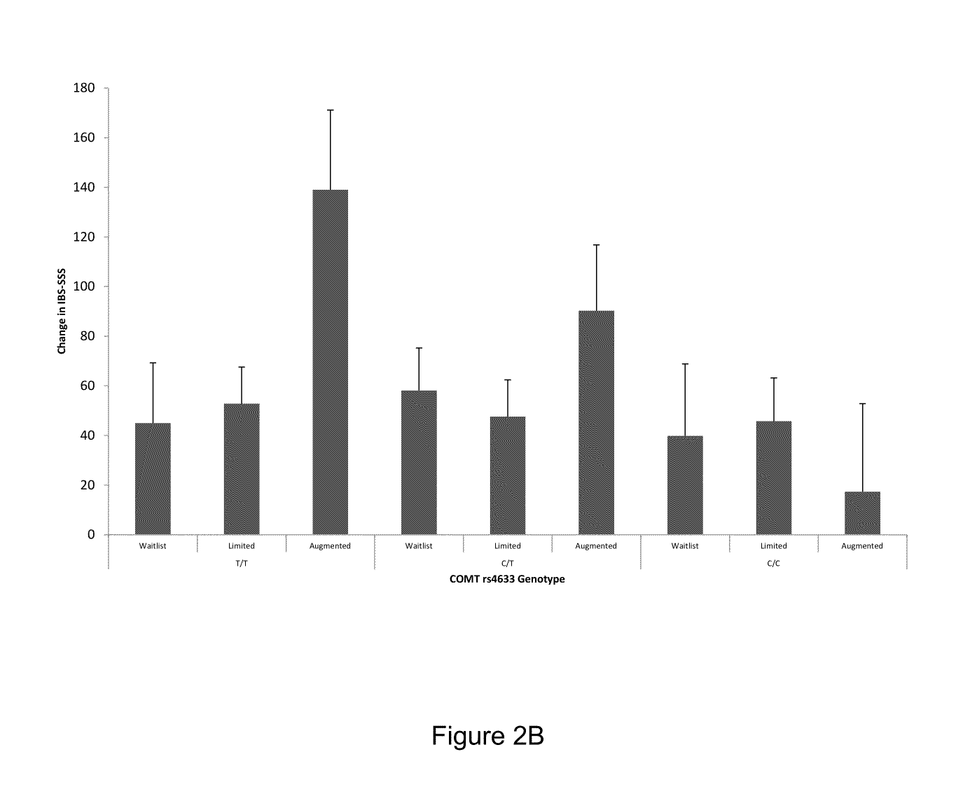

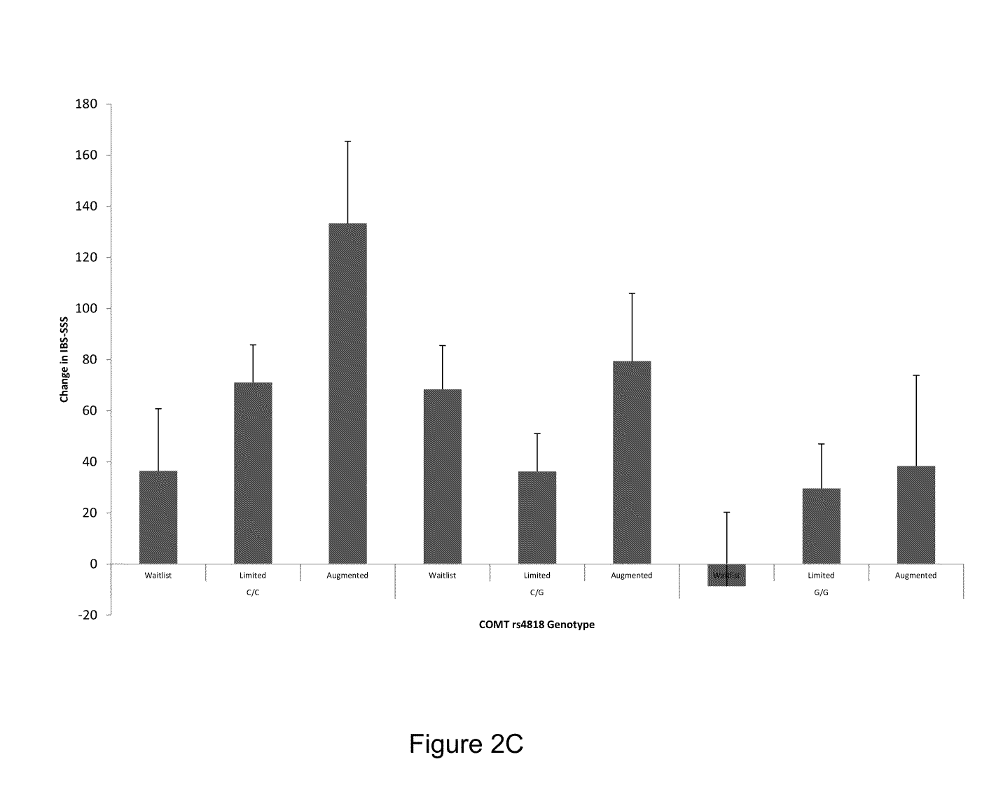

These linear effects on IBS-SSS were qualified by significant interactions between COMT genotype and treatment arm (beta=0.17; p=0.035)(FIGS. 2A-2C). COMT predicted responses in only the augmented placebo arm. In response to a high dose placebo treatment, patients homozygous for met alleles (Met/Met), showed the greatest improvement; Val homozygotes (Val/Val) showed the least improvement and heterozygote (Val/Met) patients had an intermediate response (FIG. 2A).

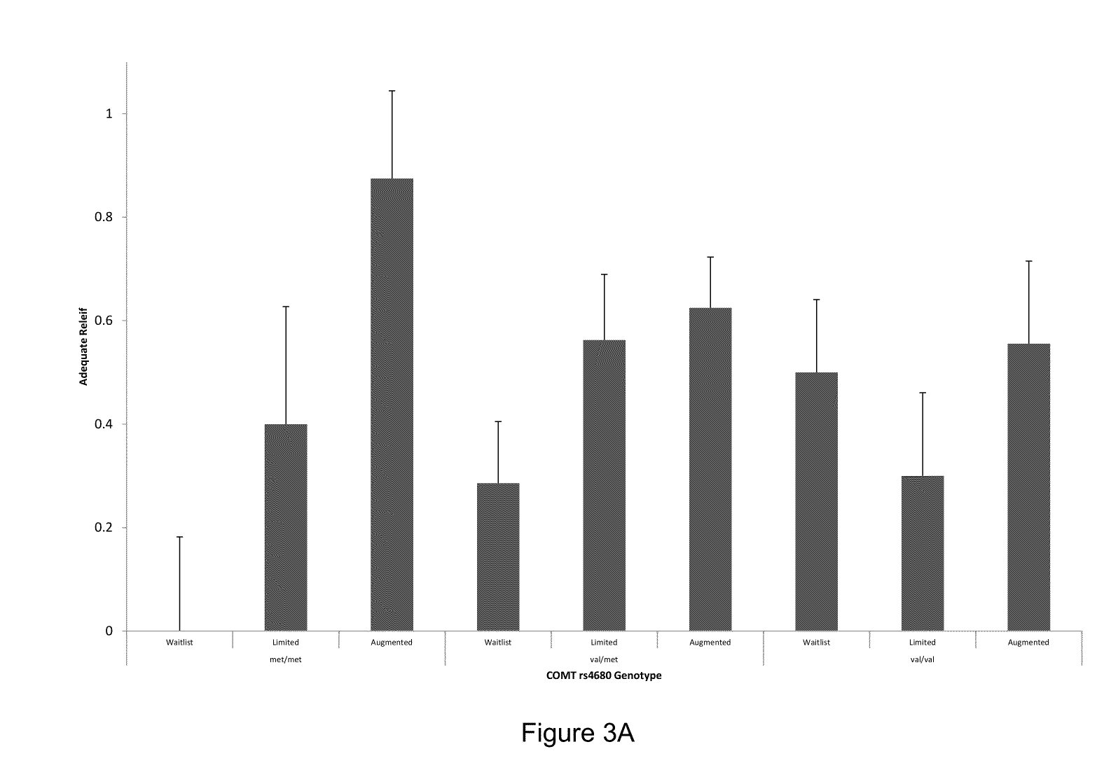

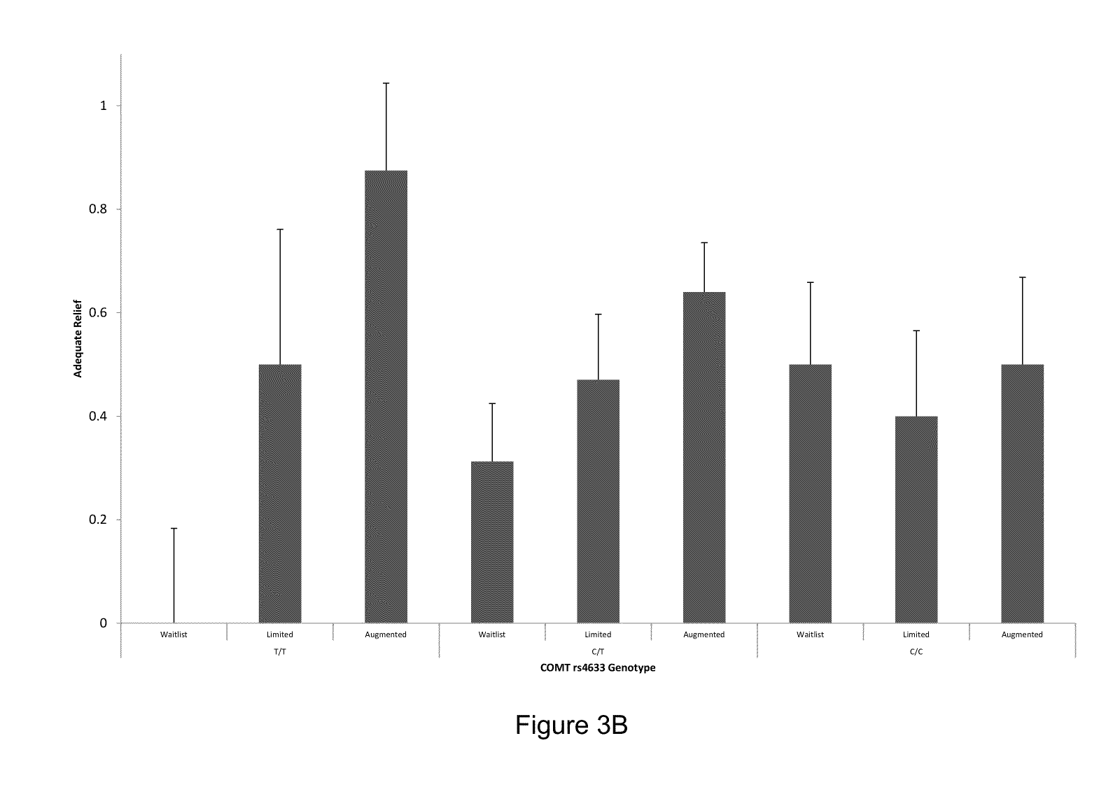

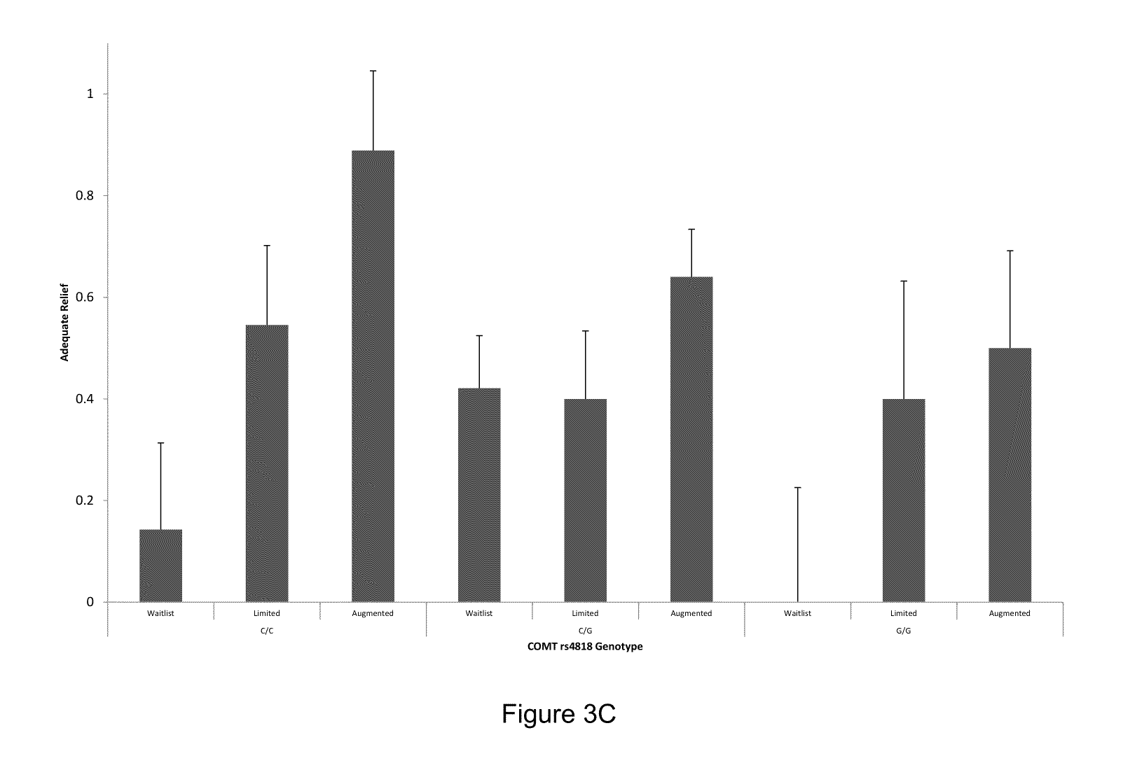

The secondary validated measure of Adequate Relief uses a single dichotomous question at 3-weeks which asked: “Over the past week have you had adequate relief of your IBS symptoms?”. The responses were coded as 0=No, I did not have Adequate Relief and 1=Yes, I had Adequate Relief. As expected from the previous study, there was a significant linear effect for treatment arm (beta=0.32, p=0.001)(FIGS. 3A-3C), in which patients in the augmented placebo arm showed most improvement and those in the waitlist arm showed the least. Although there was no main effect for COMT genotype, there was a significant interaction between COMT genotype and treatment arm (beta=0.25, p=0.009)(FIGS. 3A-3C). In the augmented placebo arm, the Met/Met patients reported an average Adequate Relief score of 0.88 compared to 0.63 for Val/Met patients and 0.56 for Val/Val patients (FIG. 3A). Conversely, in the waitlist group, Val/Val patients had an average score of 0.50, whereas the Met/Met patients had an average score of 0.00, that is no Met/Met patients on the waitlist arm reported Adequate Relief.

The other secondary validated measure was Global Improvement Scale (GIS) which asked: “Compared to the way you felt before you entered the study, have your IBS symptoms over the past 7 days been: (1)=substantially worse, (2)=moderately worse, (3)=slightly worse, (4)=no change, (5)=slightly improved, (6)=moderately improved, or (7)=substantially improved”. As in the case of IBS-SSS and Adequate Relief, there was an expected significant linear effect for treatment arm (beta=0.31, p=0.002), in which patients in the augmented placebo arm showed most improvement and those in the waitlist arm showed the least. There was a trend in improvement associated with COMT genotype (beta=0.18, p=0.063) but no interaction of COMT genotype with treatment arm (beta=0.07, p=0.477).

### Example 2

### Associated of COMT Genotype with a Placebo Response to Pain

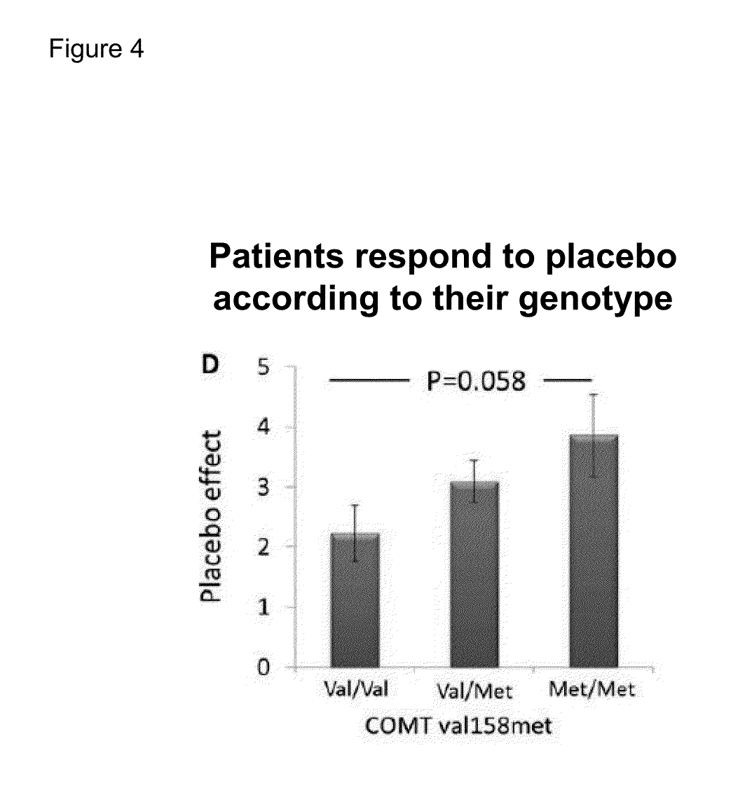

FIG. 4 is a bar graph showing the response of subjects to a pain stimulus in the presence of an analgesic placebo cue. Subjects were exposed to low or high, local heat stimulus on their forearm. During a conditioning phase the subjects learned to distinguish between a high heat and a low heat pain stimulus which was accompanied by a cue card describing the pain sensation that should be expected as “low” or “high”. In the next phase, subjects received low or high pain stimuli. In a randomized manner some of the high pain administrations were accompanied by a cue card predicting a “low” pain stimulus. This is the equivalent of a placebo analgesic. The bar graph shows the correlation of a positive response to this placebo analgesic correlated to the rs4818 COMT genotypes of the tested subjects. The p value in the 46 subjects tested is 0.058.

### Example 3

### Clinical Trial with Placebo Prescreen

Thase and colleagues (Arch Gen Psychiatry, vol 53, pp. 777-784, 1996) found that sertraline produced a clinical improvement in 59% of the 416 dysthymic patients and that the corresponding placebo treatment produced improvement in 44% of the 416 patients. The required numbers of patients for statistically significant demonstration of efficacy over placebo can be largely reduced, compared to non-prescreened trials. Prescreening patients for Met/Met and their subsequent exclusion from the clinical trial can result in placebo reduction. Using Fisher's exact test (two-tailed with α=5%), a total of 366 patients would be needed to have 80% power to detect a significant effect of medication over placebo. This example is based on the Thase sertraline (Zoloft) study. However, by identifying likely placebo responders and reducing the proportion of placebo responders to 24%, a study of only 72 patients would be needed to achieve 80% power. By identifying likely placebo responders and reducing the proportion of placebo responders to 34%, a study of only 140 patients would be needed to achieve 80% power.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### Example 4

### Prophetic Example

### Altering or Modulating a Treatment

A patient is treated for a symptom with a commercially available treatment (e.g. drug, device etc.). A medical professional tests the patient for one or more specific COMT polymorphism genotypes. The medical professional detects the presence of a COMT polymorphism genotype that indicates the patient is likely or highly likely to exhibit a placebo response if administered a placebo treatment. For example the patient tests positive for a Met158 allele and is heterozygous (e.g., Val/Met) or homozygous (e.g., Met/Met) for the polymorphism. If the patient is of high predisposition for a placebo response genotype, then the medical professional administers an additional non-pharmacological or non-device placebo treatment to the commercially available treatment response. Such “placebo” treatment could include: improvement of the therapeutic encounter (e.g., spend more “quality” time with patient (e.g., this was done during the IBS study in the group called “augmented”)), administer a placebo pill, administer non-traditional treatments for which no evidence of pharmacological efficacy is provided such as massage therapy, acupuncture, relaxation therapy and the like. Lets call all these additional options “placebo”. The formula for a response is then:

COMT placebo responder haplotype (most likely MM)+treatment (drug or device)+placebo>COMT placebo responder haplotype+treatment, or COMT non-placebo haplotype+treatment

In addition, when the patient has an augmented treatment response from treatment+placebo, the doctor may choose to taper the drug/device dose in order to reduce side-effects.

### Example 5

### Embodiments

A1. A method of selecting subjects to participate in a clinical trial comprising:
   identifying subjects with a catechol-O-methyltransferase (COMT) polymorphism, wherein the COMT polymorphism modulates a placebo effect in the subjects; and
   selecting subjects to participate in the clinical trial based on their COMT haplotype.
A2. The method of identifying subjects of embodiment 1, wherein the COMT polymorphism is at least one of rs4680, rs4818, rs6269, and rs4633.
A3. The method of identifying subjects of embodiment A1, wherein the COMT polymorphism encodes a valine/methionine haplotype.
A4. The method of identifying subjects of embodiment A1, wherein the COMT polymorphism encodes a methionine/methionine haplotype.
A5. The method of identifying subjects of embodiment A1, wherein the COMT polymorphism encodes a valine/valine haplotype.
A6. The method of identifying subjects of embodiment A1, wherein the COMT polymorphism indicates an increased likelihood the subject would exhibit a placebo effect.
A7. The method of identifying subjects of embodiment A1, wherein the subjects with the COMT polymorphism are excluded from the clinical trial.
A8. The method of identifying subjects of embodiment A1, wherein the subjects with the COMT polymorphism are included in the clinical trial.
A9. The method of identifying subjects of embodiment A1 further comprises modifying a treatment of the subjects with the COMT polymorphism are administered and administering the treatment.
A10. The method of identifying subjects of embodiment A1 further comprises increasing a number of subjects with the COMT polymorphism participating in the clinical trial, wherein the increase of subjects with the COMT polymorphism decreases a number of subjects needed for statistical significance of active treatment efficacy or intervention efficacy over placebo response.
A11. The method of identifying subjects of embodiment A1 further comprises A method of enriching high placebo responders in clinical trials for the purpose of achieving or demonstrating a synergistic treatment effect of active treatment efficacy or intervention efficacy and placebo response.
A12. A method for treating a subject in a clinical trial comprising:
   determining a catechol-O-methyltransferase (COMT) polymorphism in the subject;
   identifying a treatment for the subject based on the COMT polymorphism; and
   administering the treatment to the subject.
A13. The method of treating the subject of embodiment A12, wherein the step of determining the COMT polymorphism comprises:
   obtaining a sample from the subject and
   analyzing the sample for the COMT polymorphism.
A14. The method of treating the subject of embodiment A12, wherein the COMT polymorphism comprises at least one of rs4680, rs4818, rs6269, and rs4633.
A15. The method of treating the subject of embodiment A12, wherein the subject has an increased or decreased likelihood of exhibiting a placebo response.
A16. The method of treating the subject of embodiment A12, wherein the step of identifying the treatment comprises modulating the treatment for the subject with the COMT polymorphism and administering the modulated treatment.
A17. The method of treating the subject of embodiment A12, wherein the step of identifying the treatment comprises identifying an alternative treatment for the subject with the COMT polymorphism, and administering the treatment to the subject.
A18. The method of treating the subject of embodiment A12, wherein the step of administering the treatment comprises adjusting a dosage of the treatment administered to the subject depending on the COMT polymorphism.
A19. The method of treating the subject of embodiment A18, wherein the step of adjusting the treatment dosage comprises modulating the dosage to the subject with the COMT polymorphism.
A20. The method of treating the subject of embodiment A12, wherein the COMT polymorphism encodes a valine/methionine haplotype.
A21. The method of treating the subject of embodiment A12, wherein the COMT polymorphism encodes a methionine/methionine haplotype.
A22. The method of treating the subject of embodiment A12, wherein the COMT polymorphism encodes a valine/valine haplotype.
A23. A method of determining a treatment dosage for a subject in a clinical trial comprising:
   determining a catechol-O-methyltransferase (COMT) polymorphism in the subject; and
   modulating the treatment dosage administered to the subject based on the COMT polymorphism.
A24. The method of determining the treatment dosage of embodiment A23, wherein the step of determining the COMT polymorphism comprises:
   obtaining a sample from the subject and
   analyzing the sample for the COMT polymorphism.
A25. The method of determining the treatment dosage of embodiment A23, wherein the COMT polymorphism is at least one of rs4680, rs4818, rs6269, and rs4633.
A26. The method of determining the treatment dosage of embodiment A23, wherein the step of modulating the treatment dosage administered comprises identifying an alternative treatment for the subject with the COMT polymorphism.
A27. The method of determining the treatment dosage of embodiment A23, wherein the subject with the COMT polymorphism has an increased likelihood of exhibiting a placebo response.
A28. The method of determining the treatment dosage of embodiment A27, wherein the step of modulating the treatment dosage comprises increasing the dosage for the subject with the COMT polymorphism.
A29. The method of determining the treatment dosage of embodiment A27, wherein the step of modulating the treatment dosage comprises decreasing the dosage for the subject with the COMT polymorphism.
A30. The method of determining the treatment dosage of embodiment A27, wherein the step of modulating the treatment dosage comprises identifying an alternative treatment for the subject with the COMT polymorphism.
A31. A protein or peptide assay for determining a catechol-O-methyltransferase (COMT) polymorphism in a subject.
A32. The assay of embodiment A31 comprising at least one antibody specific for the COMT polymorphism.
A33. The assay of embodiment A31 comprising reagents for measuring catechol-O-methyltransferase enzymatic activity.
A34. The assay of embodiment A31 comprising reagents for analyzing catechol-O-methyltransferase protein.
B1. A method of initiating and/or conducting a clinical trial comprising:
   a) obtaining one or more samples from each of a plurality of human subjects considered as participants for the clinical trial;
   b) analyzing the one or more samples obtained, wherein the analyzing comprises determining the presence or absence of a catechol-O-methyltransferase polymorphism for each of the plurality of human subjects;
   c) segregating the plurality of human subjects into two or more subgroups according to the analysis in b), wherein at least one of the subgroups is designated to receive an experimental treatment.
C1. A method of selecting a sub-group of human subjects likely to have a placebo response comprising:
   a) detecting the presence or absence of at least one catechol-O-methyltransferase polymorphism in a plurality of human subjects, wherein the presence of the at least one catechol-O-methyltransferase polymorphism in a subject indicates the subject is likely to have a placebo response, and
   c) selecting a sub-group of human subjects from the plurality of human subjects wherein the sub-group of human subjects comprises the presence of the catechol-O-methyltransferase polymorphism.
C2. The method of embodiment C1, further comprising evaluating the efficacy of an experimental treatment to reduce or alleviate at least one subjective symptom for each of the subgroup of human subjects selected.
C3. The method of embodiment C1 or C2, wherein the plurality of human subjects consists of candidates for a placebo-controlled clinical trial.
C4. The method of any one of embodiments C1 to C3, wherein the presence of the at least one catechol-O-methyltransferase polymorphism is associated with a placebo effect.
C5. The method of any one of embodiments C1 to C4, wherein the catechol-O-methyltransferase polymorphism comprises a single nucleotide polymorphism in a catechol-O-methyltransferase gene.
C6. The method of any one of embodiments C1 to C5, wherein the catechol-O-methyltransferase polymorphism comprises at least one of an rs4680, rs4818, rs6269 and rs4633 polymorphism.
C7. The method of any one of embodiments C1 to C6, wherein detecting the presence or absence of a catechol-O-methyltransferase polymorphism comprises determining the presence or absence of the polymorphism in one or more alleles of a catechol-O-methyltransferase gene.
C8. The method of any one of embodiments C1 to C7, wherein detecting the presence or absence of a catechol-O-methyltransferase polymorphism comprises determining an amino acid substitution in a catechol-O-methyltransferase protein.
C9. The method of embodiment C8, wherein the amino acid substitution is a valine 158 to methionine substitution.
C10. The method of any one of embodiments C1 to C7, wherein the catechol-O-methyltransferase polymorphism comprises an rs4680 polymorphism.
D1. A method of determining a treatment dosage comprising:
   determining a catechol-O-methyltransferase (COMT) polymorphism in a subject; and
   modulating the treatment dosage administered to the subject based on the COMT polymorphism.
D2. The method of determining the treatment dosage of embodiment D1, wherein the step of determining the COMT polymorphism comprises:
   obtaining a sample from the subject and
   analyzing the sample for the COMT polymorphism.
D3. The method of determining the treatment dosage of embodiment D1, wherein the step of modulating the treatment dosage administered comprises identifying an alternative treatment for the subject with the COMT polymorphism.
D4. The method of determining the treatment dosage of embodiment D1, wherein the subject with the COMT polymorphism has an increased or decreased likelihood of exhibiting a placebo response.
D5. The method of determining the treatment dosage of embodiment D4, wherein the step of modulating the treatment dosage comprises increasing the dosage for the subject with the COMT polymorphism.
D6. The method of determining the treatment dosage of embodiment D4, wherein the step of modulating the treatment dosage comprises decreasing the dosage for the subject with the COMT polymorphism.
D7. The method of determining the treatment dosage of embodiment D1, wherein the subject is suspected of having, or is at risk for developing at least one of irritable bowel syndrome, diabetes, cardiovascular disease.
D8. The method of determining the treatment dosage of embodiment D1, wherein the COMT polymorphism comprises at least one of rs4680, rs4818, rs6269, and rs4633.
D9. A method for treating a subject comprising:
   determining a catechol-O-methyltransferase (COMT) polymorphism in the subject;
   identifying a treatment for the subject based on the COMT polymorphism; and
   administering the treatment to the subject.
D10. The method of treating the subject of embodiment D9, wherein the subject has, is suspected of having, or is at risk for developing a disorder at least one of irritable bowel syndrome; diabetes; and a cardiovascular disease.
D11. The method of treating the subject of embodiment D9, wherein the step of determining the COMT polymorphism comprises:
   obtaining a sample from the subject and
   analyzing the sample for the COMT polymorphism.
D12. The method of treating the subject of embodiment D9, wherein the COMT polymorphism comprises at least one of rs4680, rs4818, rs6269, and rs4633.
D13. The method of treating the subject of embodiment D9, wherein the subject has an increased or decreased likelihood of exhibiting a placebo effect.
D14. The method of treating the subject of embodiment D9, wherein the step of identifying the treatment comprises modulating the treatment for the subject with the COMT polymorphism and administering the modulated treatment.
D15. The method of treating the subject of embodiment D9, wherein the step of identifying the treatment comprises identifying an alternative treatment for the subject with the COMT polymorphism, and administering the treatment to the subject.
D16. The method of treating the subject of embodiment D9, wherein the step of administering the treatment comprises adjusting a dosage of the treatment administered to the subject depending on the COMT polymorphism.
D17. The method of treating the subject of embodiment D16, wherein the step of adjusting the dosage treatment comprises increasing or decreasing the dosage to the subject with the COMT polymorphism.
D18. The method of treating the subject of embodiment D9, wherein the COMT polymorphism encodes a valine/methionine haplotype.
D19. The method of treating the subject of embodiment D9, wherein the COMT polymorphism encodes a methionine/methionine haplotype.
D20. The method of treating the subject of embodiment D9, wherein the COMT polymorphism encodes a valine/valine haplotype.
D21. A protein or peptide assay for determining a catechol-O-methyltransferase (COMT) polymorphism in a subject.
D22. The assay of embodiment D21 comprising at least one antibody specific for the COMT polymorphism.
D23. The assay of embodiment D21 comprising reagents for measuring catechol-O-methyltransferase enzymatic activity.
D24. The assay of embodiment D21 comprising reagents for analyzing catechol-O-methyltransferase protein.

What is claimed is:

1. A method of selecting a sub-group of human subjects comprising:
a) detecting the presence or absence of a catechol-O-methyltransferase polymorphism in a sup-population of human subjects; and
b) selecting a sub-group of human subjects from the sup-population wherein the sub-group comprises the presence of the catechol-O-methyltransferase polymorphism.

2. The method of claim 1, comprising:
a) distributing the human subjects of the sub-population into at least two study groups; and
b) administering a placebo treatment to one or more of the at least two study groups.

3. The method of claim 2, comprising distributing the human subjects of the sub-group about equally among the at least two study groups.

4. The method of claim 2, comprising excluding the human subjects of the sub-group from the at least two study groups.

5. The method of claim 2, comprising evaluating the efficacy of an experimental treatment to reduce or alleviate a symptom.

6. The method of claim 5, wherein the symptom is a subjective symptom.

7. The method of claim 2, comprising administering an experimental treatment to one or more of the at least two study groups, wherein the experimental treatment is configured to treat a symptom.

8. The method of claim 7, wherein the symptom is a subjective symptom.

9. The method of claim 7, wherein the placebo treatment and the experimental treatment are administered to different study groups

10. The method of claim 1, wherein the sub-population of human subjects are candidates for a placebo-controlled clinical trial.

11. The method of claim 1, wherein the presence of the catechol-O-methyltransferase polymorphism is associated with a placebo effect.

12. The method of claim 1, wherein the presence of the catechol-O-methyltransferase polymorphism in a subject indicates the subject is likely to have a placebo response.

13. The method of claim 1, wherein the catechol-O-methyltransferase polymorphism comprises a single nucleotide polymorphism in a catechol-O-methyltransferase gene.

14. The method of claim 13, wherein the single nucleotide polymorphism is in one or more alleles of a catechol-O-methyltransferase gene.

15. The method of claim 13, wherein the single nucleotide polymorphism comprises at least one of an rs4680, rs4818, rs6269 and rs4633 polymorphism.

16. The method of claim 15, wherein the single nucleotide polymorphism is an rs4680 polymorphism.

17. The method of claim 16, wherein the sub-group comprising the presence of the rs4680 polymorphism is homozygous for an A allele of the rs4680 polymorphism.

18. The method of claim 16, wherein the sub-group comprising the presence of the rs4680 polymorphism is heterozygous for an A allele of the rs4680 polymorphism.

19. The method of claim 1, wherein the detecting comprises detecting an amino acid substitution in a catechol-O-methyltransferase polypeptide.

20. The method of claim 19, wherein the amino acid substitution comprises a valine 158 to methionine substitution.

21. A method for treating a subject comprising:
a) determining the presence or absence of a catechol-O-methyltransferase (COMT) polymorphism in the subject;
b) identifying a treatment for the subject based on the presence or absence of the COMT polymorphism; and
c) administering the treatment to the subject.

22. The method of claim 21, wherein the subject has, is suspected of having, or is at risk for developing a disorder selected form irritable bowel syndrome, schizophrenia, pain, diabetes, and a cardiovascular disease.

23. The method of claim 21, wherein the determining the presence or absence of a catechol-O-methyltransferase (COMT) polymorphism comprises:
a) obtaining a sample from the subject and
b) analyzing the sample for the COMT polymorphism.

24. The method of claim 21, wherein the COMT polymorphism comprises at least one of rs4680, rs4818, rs6269, and rs4633.

25. The method of claim 21, wherein the presence of a COMT polymorphism indicates the subject has an increased likelihood of exhibiting a placebo effect.

26. The method of claim 21, wherein identifying the treatment comprises modulating the treatment for the subject with the COMT polymorphism and administering the modulated treatment.

27. The method of claim 21, wherein administering the treatment comprises administering a placebo treatment to the subject.

28. The method of claim 21, wherein the presence of the COMT polymorphism comprises a presence of an A allele of an rs4680 polymorphism.

29. The method of claim 28, wherein the subject is homozygous or heterozygous for the A allele.

30. The method of claim 21, wherein the presence of COMT polymorphism encodes a valine/valine haplotype.
